(19) United States
(12) Patent Application Publication
(10) Pub. No.: US 2026/0060840 A1
(43) Pub. Date: Mar. 5, 2026

(54) Real-Time Instrument Position Estimation and Guidance for Ophthalmic Surgery

(71) Applicant: Alcon Inc., Fort Worth, TX (US)

(72) Inventors: Zhuoran Wu, Rancho Cucamonga, CA (US); Lu Yin, Keller, TX (US); Vignesh Suresh, Woodland Hills, CA (US); Ramesh Sarangapani, Coppell, TX (US); Joseph Richard Weatherbee, San Clemente, CA (US)

(21) Appl. No.: 18/820,245

(22) Filed: Aug. 29, 2024

(51) Int. Cl. A61F 9/00; A61F 9/007

(52) U.S. Cl. CPC A61F 9/0026; A61F 9/007

(57) ABSTRACT

Real-time instrument position estimation and guidance for ophthalmic surgery are described. An ophthalmic surgical system may include a first camera to obtain a first image feed of an ophthalmic surgical procedure and a second camera to obtain a second image feed of the ophthalmic surgical procedure. The ophthalmic surgical system may further include a real-time instrument position estimation module implemented in a non-transitory computer-readable storage medium and configured to determine, in real-time, a relative position of an instrument feature with respect to an anatomical feature of an eye based on the first image feed and the second image feed during the ophthalmic surgical procedure. The real-time instrument position estimation module may be further configured to output, in real-time, a surgical guidance based on the relative position of the instrument feature with respect to the anatomical feature of the eye during the ophthalmic surgical procedure.

### BACKGROUND

A variety of diseases or conditions associated with an eye may be treated through ophthalmic surgical procedures. Examples of ophthalmic surgical procedures include vitreoretinal surgery, cataract surgery, glaucoma surgery, laser eye surgery (LASIK), and the like. For example, vitreoretinal surgery is a type of eye surgery that treats problems with the retina or the vitreous and may be performed for treating conditions such as diabetic traction retinal detachment, diabetic vitreous hemorrhage, macular hole, retinal detachment, epimacular membrane, and many other ophthalmic conditions. As another example, cataract surgery involves emulsifying a crystalline lens with an ultrasonic handpiece and aspirating it from the eye. An intraocular lens (IOL) is then implanted in the posterior lens capsule of the eye. During various ophthalmic surgical procedures such as those mentioned above, imprecise manipulation of surgical tools may negatively impact the outcome, efficiency, and effectiveness of the surgery.

### SUMMARY

Real-time instrument position estimation and guidance for ophthalmic surgery are described. An ophthalmic surgical system may include a first camera to obtain a first image feed of an ophthalmic surgical procedure and a second camera to obtain a second image feed of the ophthalmic surgical procedure. The ophthalmic surgical system may further include a real-time instrument position estimation module implemented in a non-transitory computer-readable storage medium and configured to determine, in real-time, a relative position of an instrument feature with respect to an anatomical feature of an eye based on the first image feed and the second image during the ophthalmic surgical procedure. The real-time instrument position estimation module may be further configured to output, in real-time, a surgical guidance based on the relative position of the instrument feature with respect to the anatomical feature of the eye during the ophthalmic surgical procedure.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures. Entities represented in the figures are indicative of one or more entities and thus, reference is made interchangeably to single or plural forms of the entities in the discussion.

FIG. 1 is an illustration of an environment in an example implementation that is operable to employ a real-time instrument position estimation and guidance for ophthalmic surgery as described herein.

FIG. 2 depicts a cross-section of an eye.

FIG. 3 depicts an example implementation of the real-time instrument position estimation module of FIG. 1 in greater detail.

FIG. 4 depicts a non-limiting example of using pre-op patient measurements to determine physical measurement units from pixel values in the context of cataract surgery.

FIG. 5 depicts an illustrative example overview of calculating the relative depth during cataract surgery.

FIG. 6 depicts an example of real-time instrument position estimation and guidance during cataract surgery.

FIG. 7 depicts an example of real-time instrument position estimation and guidance during vitreoretinal surgery.

FIG. 8 depicts an example of real-time instrument position estimation and guidance during a subretinal injection.

FIG. 9 depicts an example of real-time instrument position estimation and guidance during a minimally invasive glaucoma surgery.

FIG. 10 depicts an example procedure in which real-time instrument position estimation and guidance for ophthalmic surgery is performed.

FIG. 11 illustrates an example system including various components of an example device that can be implemented as any type of computing device as described and/or utilized with reference to FIGS. 1-10 to implement the techniques described herein.

### DETAILED DESCRIPTION

### Overview

Eye surgeons operate instruments inside a patient's eye, which is a relatively small, confined space. Various visualization systems provide surgeons with digitally enhanced views of the eye and a position of a surgical instruments therein. However, it may still be difficult for the surgeon to accurately assess the instrument position, particularly in terms of depth. Enhanced information regarding instrument position, including relative depth information, may increase procedure precision and improve patient outcomes.

Accordingly, real-time instrument position estimation and guidance for ophthalmic surgery is described herein. By way of example, an ophthalmic surgical system employs an object-based approach to efficiently determine the relative three-dimensional position of a targeted instrument feature with respect to a targeted anatomical feature of the eye. By identifying and tracking specific features for calculating relative disparity and the relative three-dimensional position therefrom, the ophthalmic surgical system achieves fast computation times that are suitable for real-time applications.

In accordance with the described techniques, the ophthalmic surgical system obtains a stereoscopic image feed of an ophthalmic surgical procedure from two cameras with different viewpoints. An object recognition algorithm may detect and identify the targeted instrument feature and anatomical feature in both image feeds. A position estimation algorithm may then determine the spatial locations of these features. Using these spatial locations, a disparity calculation algorithm may compute the relative disparity between the instrument feature and the anatomical feature. This relative disparity, combined with camera configuration data such as focal length and optical channel separation, may enable a depth estimation algorithm to calculate the relative depth of the instrument feature with respect to the anatomical feature.

The described techniques facilitate the generation of comprehensive surgical guidance. By way of example, based on the calculated relative position of the instrument feature, the ophthalmic surgical system can output real-time visual overlays, numerical depth values, or color-coded warnings to guide the surgeon. This immediate and/or instantaneous feedback allows for more accurate instrument positioning and can help the surgeon avoid delicate eye structures. Alternatively, or in addition, the calculated relative position of the instrument feature can be used to generate commands used to drive the instrument in robot-assisted surgical applications.

The object-based approach of the techniques described herein can offer one or more of several technical advantages. By focusing on specific features of interest rather than processing every pixel in the images, for instance, the approach described herein significantly reduces computational load compared to pixel-by-pixel disparity calculation methods. This efficiency allows for real-time position estimation and guidance, as delays could cause a discrepancy between the estimated position and the actual, updated position of the instrument. Additionally, the use of pre-operative measurements and camera configuration data allows for the calculations to be adapted to the patient's anatomy and also facilitates the conversion of pixel-based measurements to physical units, thus providing surgeons with intuitive and actionable position information.

Furthermore, the techniques described herein are adaptable to various ophthalmic surgical procedures, including cataract surgery, vitreoretinal surgery, and subretinal injections. The object recognition algorithm can be configured to detect different instrument features and anatomical landmarks relevant to each procedure, for instance. This flexibility, combined with the real-time capabilities of the described approach, enables more precise and surgical interventions across a range of ophthalmic applications.

In some aspects, the techniques described herein relate to an ophthalmic surgical system including: a first camera to obtain a first image feed of an ophthalmic surgical procedure; a second camera to obtain a second image feed of the ophthalmic surgical procedure, and a real-time instrument position estimation module implemented in a non-transitory computer-readable storage medium and configured to perform operations including: determining, in real-time, a relative position of an instrument feature with respect to an anatomical feature of an eye based on the first image feed and the second image feed during the ophthalmic surgical procedure, and outputting, in real-time, a surgical guidance based on the relative position of the instrument feature with respect to the anatomical feature of the eye during the ophthalmic surgical procedure.

In some aspects, the techniques described herein relate to an ophthalmic surgical system, wherein determining, in real-time, the relative position of the instrument feature with respect to the anatomical feature of the eye based on the first image feed and the second image feed during the ophthalmic surgical procedure includes: determining respective spatial locations of the instrument feature and the anatomical feature in the first image feed and the second image feed; calculating a relative disparity between the instrument feature and the anatomical feature based on the respective spatial locations; and estimating a relative depth of the instrument feature with respect to the anatomical feature based on the relative disparity and a configuration of the first camera and the second camera.

In some aspects, the techniques described herein relate to an ophthalmic surgical system, wherein determining the respective spatial locations of the instrument feature and the anatomical feature in the first image feed and the second image feed includes: identifying the instrument feature and the anatomical feature in the first image feed and the second image feed using a machine learning model that is trained to detect the instrument feature and the anatomical feature in the respective images.

In some aspects, the techniques described herein relate to an ophthalmic surgical system, wherein calculating the relative disparity between the instrument feature and the anatomical feature includes: determining respective pixel coordinate positions of the instrument feature and the anatomical feature based on the respective spatial locations; calculating a unit per pixel value based on pre-operative measurements of the eye and a pixel measurement of the anatomical feature; and calculating the relative disparity based on a first difference between the respective coordinate positions of the instrument feature in the first image feed and the second image feed, a second difference between the respective coordinate positions of the anatomical feature in the first image feed and the second image feed; and the unit per pixel value.

In some aspects, the techniques described herein relate to an ophthalmic surgical system, wherein the configuration of the first camera and the second camera includes a focal length and an optical channel separation.

In some aspects, the techniques described herein relate to an ophthalmic surgical system, wherein the relative position includes one or more of a relative depth of the instrument feature with respect to the anatomical feature of the eye, a relative three-dimensional position of the instrument feature with respect to the anatomical feature of the eye, and a planar distance between the instrument feature and the anatomical feature of the eye.

In some aspects, the techniques described herein relate to an ophthalmic surgical system, wherein the surgical guidance includes a position indication.

In some aspects, the techniques described herein relate to an ophthalmic surgical system, wherein outputting the surgical guidance further includes: generating a visual indicator of the position indication based on the relative position of the instrument feature with respect to at least one threshold that defines a targeted position of the instrument feature relative to the anatomical feature; and outputting the visual indicator via a display.

In some aspects, the techniques described herein relate to an ophthalmic surgical system, wherein the surgical guidance includes an instrument adjustment provided to an instrument having the instrument feature, the instrument adjustment including a command to adjust one or more of an angle, a depth, a plane, a speed, a directionality, and a parameter setting of the instrument.

In some aspects, the techniques described herein relate to an ophthalmic surgical system, wherein: the ophthalmic surgical procedure is cataract surgery; the instrument feature is a tip of a phacoemulsification probe; and the anatomical feature is the limbus.

In some aspects, the techniques described herein relate to an ophthalmic surgical system, wherein: the ophthalmic surgical procedure is a vitrectomy; the instrument feature is a tip of a vitrectomy probe; and the anatomical feature is a retinal feature.

In some aspects, the techniques described herein relate to an ophthalmic surgical system, wherein: the ophthalmic surgical procedure is a subretinal injection; the instrument feature is a tip of an injection canula; and the anatomical feature is a retinal feature.

In some aspects, the techniques described herein relate to an ophthalmic surgical system, wherein: the ophthalmic surgical procedure is minimally invasive glaucoma surgery; the instrument feature is a tip of a stent implanter; and the anatomical feature is Trabecular meshwork.

In some aspects, the techniques described herein relate to a method for an ophthalmic surgical procedure, including: obtaining, via an intra-operative imaging system, images of an eye during the ophthalmic surgical procedure, the intra-operative imaging system including a first camera and a second camera having different viewpoints of the ophthalmic surgical procedure; processing the images of the ophthalmic surgical procedure in real-time to determine a relative position of a targeted instrument feature with respect to a targeted anatomical feature of the eye; and outputting a surgical guidance in real-time based on the relative position of the targeted instrument feature with respect to the targeted anatomical feature of the eye.

In some aspects, the techniques described herein relate to a method, wherein processing the images of the ophthalmic surgical procedure in real-time to determine the relative position of the targeted instrument feature with respect to the targeted anatomical feature of the eye includes: detecting, via an object recognition algorithm, the targeted instrument feature and the targeted anatomical feature of the eye in the images of the ophthalmic surgical procedure as the images of the ophthalmic surgical procedure are obtained; estimating, via a position estimation algorithm, respective spatial locations of the targeted instrument feature and the targeted anatomical feature of the eye in the images of the ophthalmic surgical procedure in response to the detecting; calculating, via a disparity calculation algorithm, a relative disparity between the targeted instrument feature and the targeted anatomical feature of the eye based on the respective spatial locations and pre-operative eye measurement data; and estimating, via a depth estimation algorithm, a relative depth of the targeted instrument feature with respect to the targeted anatomical feature of the eye based on the relative disparity, an optical camera separation between the first camera and the second camera, and a focal length of the first camera and the second camera.

In some aspects, the techniques described herein relate to a method, wherein the pre-operative eye measurement data include a measurement of the targeted anatomical feature of the eye in a physical measurement unit, and wherein calculating, via the disparity calculation algorithm, the relative disparity between the targeted instrument feature and the targeted anatomical feature of the eye based on the respective spatial locations includes: calculating a first disparity of the targeted instrument feature based on the respective spatial locations of the targeted instrument feature in a first image obtained by the first camera and a second spatial location of the targeted instrument feature in a second image obtained by the second camera, the first image and the second image obtained simultaneously; calculating a second disparity of the targeted instrument feature based on the respective spatial locations of the targeted anatomical feature in the first image and the second image; calculating the relative disparity in pixel values based on a difference between the first disparity and the second disparity; and converting the pixel values to the physical measurement unit based on a pixel measurement of the targeted anatomical feature in the images and the measurement of the targeted anatomical feature in the physical measurement unit.

In some aspects, the techniques described herein relate to a method, wherein the object recognition algorithm includes a machine learning model trained to detect the targeted instrument feature and the targeted anatomical feature in the images based on the ophthalmic surgical procedure being performed.

In some aspects, the techniques described herein relate to a method, wherein the surgical guidance includes an overlay on the images regarding the relative position of the targeted instrument feature with respect to the targeted anatomical feature of the eye and with further respect to an adjustable threshold range.

In some aspects, the techniques described herein relate to an ophthalmic surgical system including: an intra-operative imaging system to obtain images of an ophthalmic surgical procedure performed on an eye, the intra-operative imaging system including a first camera and a second camera; and a real-time instrument position estimation module implemented in a non-transitory computer-readable storage medium and configured to perform operations including: determining, in real-time as the images are obtained, a relative position of an instrument feature with respect to an anatomical feature of the eye based on the images, pre-operative measurements of the eye, and an arrangement of the first camera and the second camera in the intra-operative imaging system; and outputting, in real-time as the images are obtained, a surgical guidance based on the relative position of the instrument feature with respect to the anatomical feature of the eye.

In some aspects, the techniques described herein relate to a system, wherein the surgical guidance includes a visualization of the relative position of the instrument feature with respect to the anatomical feature of the eye.

### Example Environment

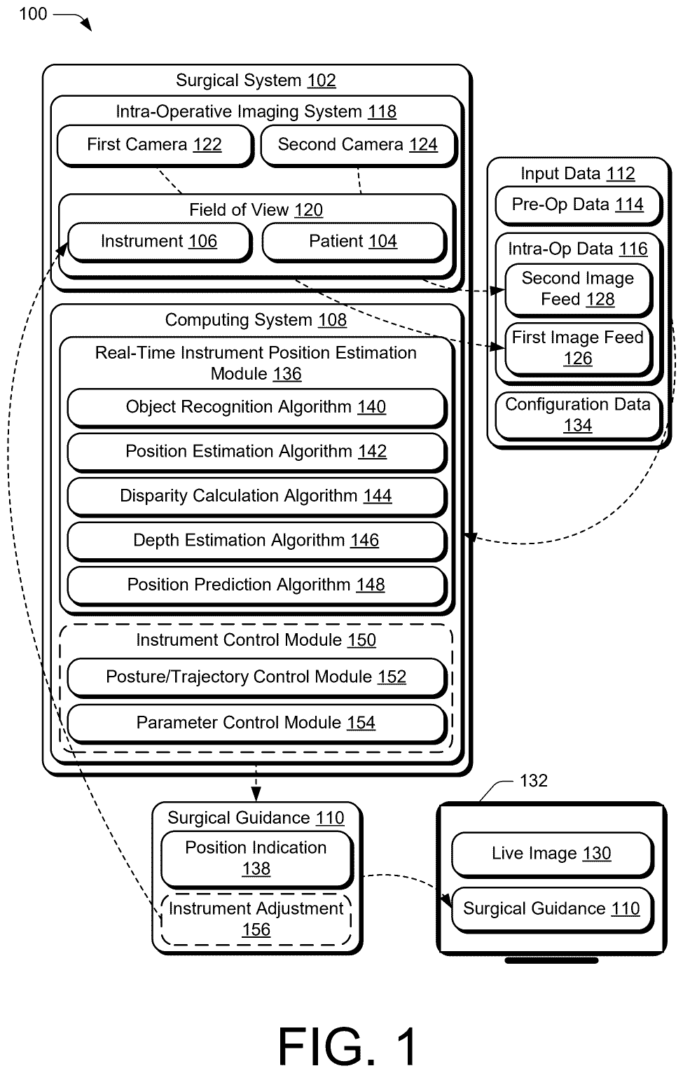

FIG. 1 is an illustration of an environment 100 in an example implementation that is operable to employ a real-time instrument position estimation and guidance for ophthalmic surgery as described herein. The illustrated environment 100 includes a surgical system 102. The surgical system 102 may be a system configured to perform one or more ophthalmic surgical procedures, such as cataract surgery, vitreoretinal surgery, and the like, on a patient 104 using an instrument 106.

In at least one implementation, the surgical system 102 includes a computing system 108, which is representative of one or more co-located or non-co-located systems that execute instructions to generate a surgical guidance 110. As will be elaborated herein below, the surgical guidance 110 provides surgical assistance (e.g., to a surgeon) via enhanced visualization and/or messaging. Alternatively, or in addition, the surgical guidance 110 may directly control the instrument 106, such as by adjusting a robotic system driving the instrument 106.

In at least one implementation, the computing system 108 generates the surgical guidance 110 based on input data 112. The input data 112 includes pre-operative (e.g., “pre-op”) data 114, configuration data and intra-operative (e.g., “intra-op”) data 116. The pre-op data 114, for instance, may include information about the patient, including data that may be received from a database, such as an electronic medical record (EMR) database for storing patient history information, and/or data that is generated and provided by a pre-op imaging system regarding the eye of the patient 104. For example, the pre-op data 114 may include one or more relevant physiological measurements and/or images related to the eye. As an example, the pre-op data 114 may include pre-op images of one or more optical components of the eye (e.g., the retina, crystalline lens, cornea, etc.). Alternatively, or in addition, the pre-op data 114 may include pre-op measurements of the patient 104 regarding, e.g., the axial length of the eye, corneal curvature, anterior chamber depth, white-to-white diameter (e.g., width) of the limbus, lens thickness, effective lens position, and so forth, as well as measurements relating to eye diseases and other conditions.

The intra-op data 116, for instance, may include information obtained and/or generated during the surgical procedure performed on the patient 104. For example, the intra-op data 116 may include data input into the computing system 108 (e.g., by a user, such as the surgeon or another medical practitioner) and/or generated by the surgical system 102. As a part of this, the surgical system 102 includes an intra-operative imaging system 118 configured to obtain intra-operative images as the surgical procedure is performed on the patient 104. By way of example, the intra-operative imaging system 118 may be a microscope, such as a digital microscope, a surgical microscope, or the like.

In at least one implementation, the intra-operative imaging system 118 obtains imaging data of a field of view 120 via a first camera 122 and a second camera 124. In particular, the first camera 122 obtains a first image feed 126 from a first viewing angle with respect to the field of view 120 (e.g., a left imaging channel), and the second camera 124 obtains a second image feed 128 from a second viewing angle the field of view 120 (e.g., a right imaging channel). As shown in the example environment 100 of FIG. 1, the first image feed 126 and the second image feed 128 may be included as part of the intra-op data 116. In at least one implementation, the first image feed 126 and the second image feed 128 capture live (e.g., real-time) visual data of the field of view 120. Moreover, the first camera 122 and the second image feed 128 may be configured to obtain images at the same frame rate and at substantially the same time (e.g., substantially simultaneously) so that a first frame of the first image feed 126 corresponds to a first frame of the second image feed 128, a second frame of the first image feed 126 corresponds to a second frame of the second image feed 128, and so forth. In at least one implementation, the first image feed 126 and the second image feed 128 are merged to generate a live image 130 of the view 120. The live image 130 may be displayed on a display device 132 substantially at the time of capture (e.g., without intentional delay). In at least one implementation, the live image 130 is a stereoscopic image.

The field of view 120 is representative of an area that is visible to the first camera 122 and the second camera 124, e.g., through optical lenses, to obtain the first image feed 126 and the second image feed 128, respectively. In at least one implementation, such as the example indicated in FIG. 1, the field of view 120 includes at least a portion of the instrument 106 and at least a portion of the patient 104 (e.g., an eye of the patient 104, or a portion thereof). As such, it is to be appreciated that during an ophthalmic surgical procedure, the intra-operative imaging system 118 is configured to obtain images (e.g., live images) depicting the procedure being performed on the patient 104.

In one or more implementations, the input data 112 further include configuration data 134 of the surgical system 102. By way of example, the configuration data 134 define an arrangement of the first camera 122 and the second camera 124 in the intra-operative imaging system 118, such as a focal length and an optical channel separation between the first camera 122 and the second camera 124. The configuration data 134 may be manually input by a user and/or provided to the computing system 108 by the intra-operative imaging system 118, with or without user intervention.

In at least one implementation, the computing system 108 is configured to analyze the input data 112 using a real-time instrument position estimation module 136 to generate the surgical guidance 110. A “module” may include a hardware and/or software system that operates to perform one or more functions, such as the functions that will be described below. For example, a module may include, or may be included in, a computer processor, a controller, or another logic-based device that performs operations based on instructions stored on a tangible and non-transitory computer readable storage medium, such as a computer memory. Alternatively, a module may include a hardwired device that performs operations based on hardwired logic of the device. The various modules shown in the attached figures, including FIG. 1, may represent the hardware that operates based on software or hardwired instructions, the software that directs hardware to perform the operations, or a combination thereof. The hardware may include electronic circuits that include and/or are connected to one or more logic-based devices, such as microprocessors, processors, controllers, and the like.

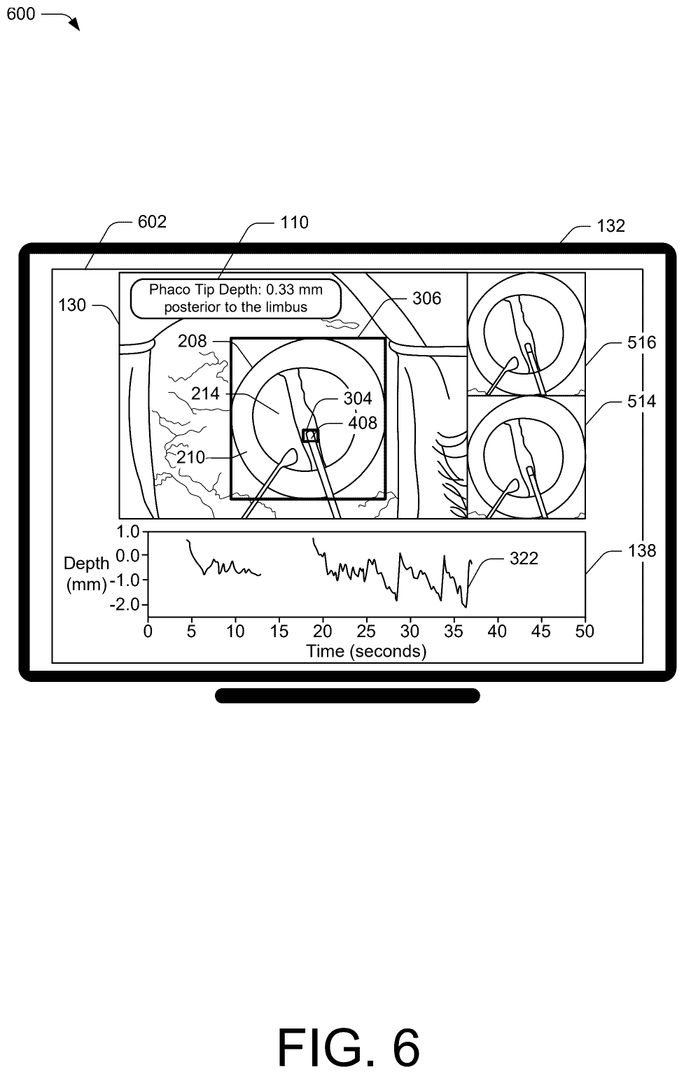

The real-time instrument position estimation module 136 includes functionality that is executed by the computing system 108 to estimate a three-dimensional (3D) position of a targeted portion of the instrument 106 based on the input data 112. The 3D position, for instance, may be a relative position of an imaged (e.g., via the intra-operative imaging system 118) and targeted portion of the instrument 106, such as relative to an imaged and targeted anatomical feature of the patient 104. The targeted anatomical feature of the patient 104 may provide a reference against which the position of the targeted portion of the instrument 106 is compared, for instance. As such, in at least one implementation, the surgical guidance 110 includes a position indication 138. The position indication 138 may include a visual message, an auditory message, and/or another type of prompt that conveys information regarding the position of the instrument 106 (e.g., the targeted portion of the instrument 106) relative to the targeted anatomical feature. Non-limiting examples of the position indication 138 are provided herein, e.g., with reference to FIGS. 6-9.

As will be further elaborated herein, the position indication 138 may be an indication of a relative three-dimensional (3D) position of the targeted portion of the instrument 106, also referred to herein as an instrument feature, relative to the targeted anatomical feature. By way of example, the position indication 138 may convey the relative depth of the instrument feature with respect to the targeted anatomical feature and/or a spatial location of the instrument feature with respect to the to the targeted anatomical feature, such as a planar distance (e.g., within a plane of the anatomical feature) between the instrument feature and the targeted anatomical feature. In at least one implementation, the position indication 138 additionally or alternatively conveys an anticipated trajectory of the instrument feature.

In order to generate the position indication 138, at least one implementation, the real-time instrument position estimation module 136 includes an object recognition algorithm 140, a position estimation algorithm 142, a disparity calculation algorithm 144, a depth estimation algorithm 146, and a position prediction algorithm 148. The object recognition algorithm 140, the position estimation algorithm 142, the disparity calculation algorithm 144, the depth estimation algorithm 146, and the position prediction algorithm 148, for instance, may represent sets of instructions for performing specific subtasks in generating the position indication 138. It is to be appreciated, however, that the real-time instrument position estimation module 136 may include more, fewer, or different algorithms for generating the position indication 138. By way of example, in at least one variation, the position prediction algorithm 148 is not included in the real-time instrument position estimation module 136, and the real-time instrument position estimation module 136 may generate the position indication 138 based on a current location of the instrument feature relative to the targeted anatomical feature without predicting or anticipating a subsequent location of the instrument feature, as will be further elaborated below.

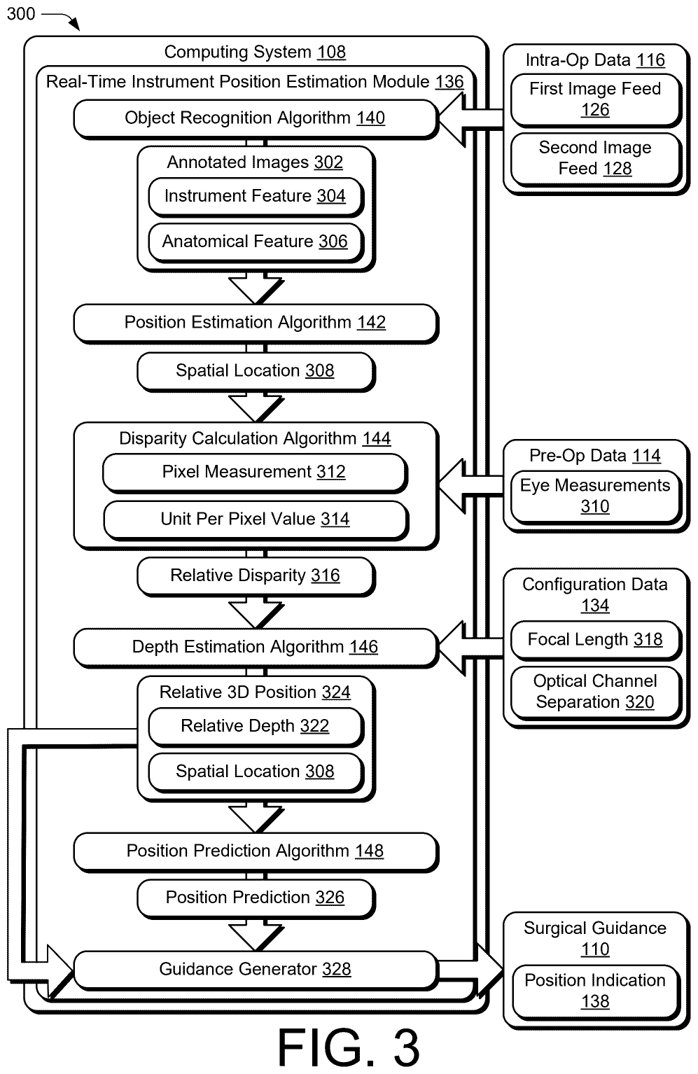

The object recognition algorithm 140, for instance, may provide computer vision functionality to the computing system 108. By way of example, the object recognition algorithm 140 may perform feature extraction (where edges, textures, and/or other key points are extracted from the first image feed 126 and the second image feed 128), object detection (e.g., where bounding region(s) of object(s) are located based on the extracted features), and classification (e.g., where the detected objects are classified based on trained categories). In at least one implementation, the object recognition algorithm 140 includes a machine learning model trained to detect the targeted portion of the instrument 106 (e.g., a particular feature or features of the instrument 106) and the targeted anatomical feature. The machine learning model may be or may include a neural network (e.g., a convolutional neural network, a deep neural network, a recurrent neural network), a support vector machine, a transformer-based model, and/or a regression model (e.g., linear, polynomial, and/or logistic regression models), just to name a few. By way of example, the machine learning model may, through a training process, learn patterns in the first image feed 126 and the second image feed 128 that are indicative of particular anatomical or instrument components. The underlying model(s) of the object recognition algorithm 140 may be trained using different approaches, such as using supervised learning, unsupervised learning, semi-supervised learning, and/or reinforcement learning. Once trained, the object recognition algorithm 140 is configured to use those one or more models to process the first image feed 126 and the second image feed 128 in real-time to detect the targeted portion of the instrument 106 and the targeted anatomical feature, as will be further described herein with respect to FIG. 3.

It is to be appreciated that the object recognition algorithm 140 may be configured as, or include, other types of models without departing from the spirit or scope of the described techniques. These types of models may be built or trained (or the model otherwise learned), respectively, using different algorithms and different data due, at least in part, to different architectures and/or learning paradigms. Accordingly, it is to be appreciated that the following discussion of object recognition algorithm 140 is applicable to a variety of different algorithms that enable object detection and classification in real-time images.

In at least one variation, for instance, the object recognition algorithm 140 receives input from the surgeon regarding which object(s) to recognize and localize in subsequent images. By way of example, rather than analyzing an entirety of the view 120, the object recognition algorithm 140 tracks user-selected object(s) in subsequent image frames of the first image feed 126 and the second image feed 128. This may enable the object recognition algorithm 140 to identify the user-selected object(s) in the subsequent image frames without specifically learning to identify and classify those object(s) through a training process.

The position estimation algorithm 142 may include a step-by-step procedure for determining spatial locations of the targeted portion of the instrument 106 and the targeted anatomical feature of the eye within the first image feed 126 and the second image feed 128. The spatial locations may be respective X and Y (e.g., horizontal and vertical) locations of the targeted portion of the instrument 106 and the targeted anatomical feature, such as X and Y coordinates of their centroids. In at least one implementation, the spatial location of the targeted portion of the instrument 106 is calibrated to the targeted anatomical feature of the eye.

The disparity calculation algorithm 144 may include a step-by-step procedure for calculating a relative disparity between the targeted portion of the instrument 106 and the targeted anatomical feature of the eye based on their spatial locations in the first image feed 126 and the second image feed 128, e.g., as identified and localized via the object recognition algorithm 140. The relative disparity, for instance, is the difference between horizontal positions of corresponding points in the first image feed 126 and the second image feed 128. In at least one implementation, the disparity calculation algorithm 144 further determines a distance (e.g., millimeter) per pixel measurement based on the pre-op data 114, such as will be further described with respect to FIG. 3.

In at least one variation, the disparity calculation algorithm 144 calculates the absolute disparity of each image pixel between the first image feed 126 and the second image feed 128. However, calculating the disparity based on the spatial locations of recognized targeted features, such as introduced above, reduces computational resources and increases computing efficiency.

The depth estimation algorithm 146 may include a step-by-step procedure for calculating a relative depth of the targeted portion of the instrument 106 (e.g., relative to the targeted anatomical feature) based on the relative disparity determined by the disparity calculation algorithm 144. The depth estimation algorithm 146, for instance, uses the configuration data 134 (e.g., the focal length and the optical channel separation) along with the relative disparity to determine the relative depth of the targeted portion of the instrument 106. This information is then used to generate the position indication 138. In at least one implementation, the position indication 138 may be overlaid on or provided beside the live image 130 on the display device 132. The position indication 138 may be customized based on user input, at least in one implementation. By way of example, the position indication 138 may indicate numerical depth values and/or percentages, colors to indicate different location zones, or any other suitable format.

The position prediction algorithm 148 may include a step-by-step procedure for tracking and anticipating the 3D position of the targeted portion of the instrument 106 over time. By way of example, the position prediction algorithm 148 may predict a future location of the targeted portion of the instrument 106 based on its position at a current image frame and positions at prior image frames. The position prediction algorithm 148, for instance, may determine a displacement of the targeted portion of the instrument 106 between sequential image frames and use this to anticipate a subsequent position (e.g., the XY position and/or depth) of the targeted portion of the instrument 106.

The computing system 108 optionally further includes an instrument control module 150. By way of example, the instrument control module 150 may be present in robot-assisted surgery applications. In at least one implementation of a robot-assisted surgery scenario, the instrument control module 150 includes a posture/trajectory control module 152 configured to control and/or adjust an angle, depth, plane, speed, and/or directionality of the instrument 106 and a parameter control module 154 configured to control and/or adjust parameter setting(s) of the instrument 106, such as a power setting, a fluid infusion rate, an aspiration flow rate, and the like. In such implementations, the surgical guidance 110 further includes an instrument adjustment 156 that is provided to the instrument 106. The instrument control module 150, for instance, may use the position indication 138 to determine the instrument adjustment 156 (e.g., posture/trajectory adjustment(s) and/or parameter adjustment(s)) that will drive the targeted portion of the instrument 106 to the targeted anatomical feature. As such, the position indication 138 may be used to guide the instrument 106 to a particular location in the eye during the surgical procedure.

In this way, the environment 100 provides a computationally efficient system for providing real-time surgical guidance to surgeons during an ophthalmic procedure. As a result, less surgeon-to-surgeon variation may be achieved while increasing an occurrence of positive patient outcomes.

To facilitate the discussion of additional details of the real-time instrument position estimation guidance for ophthalmic surgery, an overview of anatomic features of the eye will now be provided.

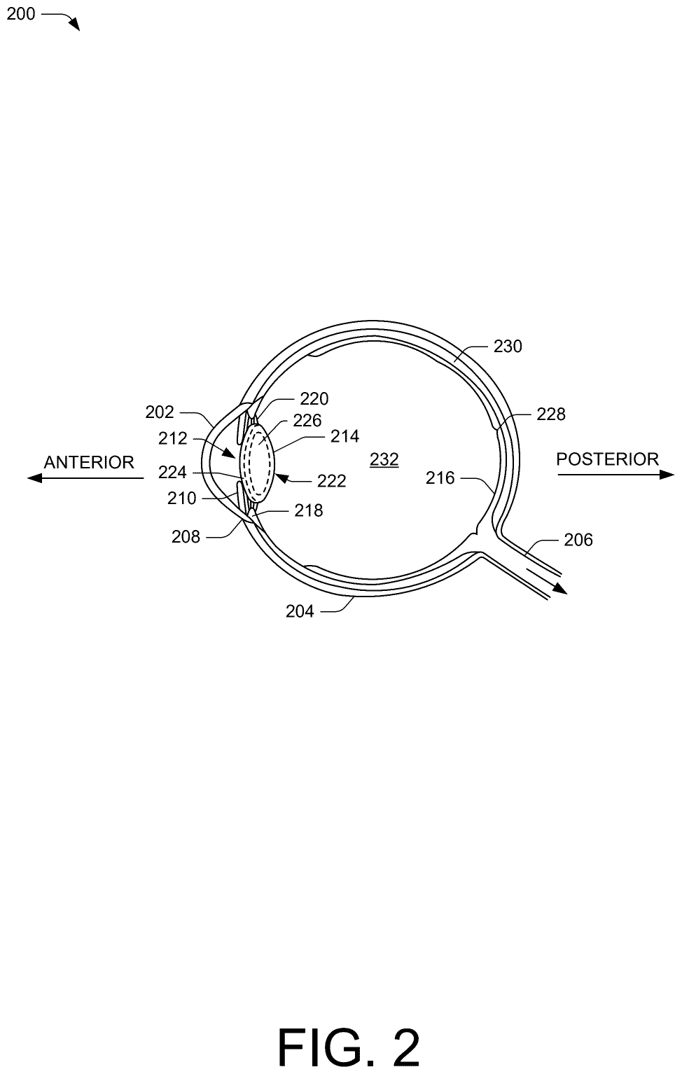

FIG. 2 depicts a cross-section of an eye 200. The eye 200 is a generally globular fused two-piece unit including a cornea 202 and sclera 204. The cornea 202 is a smaller transparent, dome-shaped front layer of the eye 200, whereas the sclera 204 is an opaque outer later that provides structural support and extends from the cornea 202 to an optic nerve 206. The cornea 202 is connected to the sclera 204 at a limbus 208. The limbus 208, for instance, is a ring that provides a transition zone between the cornea 202 and the sclera 204. The limbus 208 is typically about 11-12 millimeters (mm) in width (e.g., horizontal diameter) and 10-11 mm in height (e.g., vertical diameter), appearing elliptical in shape. The cornea 202 comprises approximately one-sixth of the outer surface area of the eye 200, whereas the sclera 204 comprises the remaining five-sixths. Moreover, the cornea 202 protrudes beyond the globe of the sclera 204 due to having a different (e.g., smaller) radius of curvature than the sclera 204.

The eye 200 further includes an iris 210. The iris 210 is the colored part of the eye 200 and surrounds a pupil 212. The iris 210 controls the size of the pupil 212, which is the central opening that allows light to enter the eye. The pupil 212 adjusts the size of the pupil 212 in response to light intensity, such as by contracting to reduce the size of the pupil 212 in response to bright light and dilating to increase the size of the pupil 212 in response to dim light. The iris 210 and the pupil 212 are seen instead of the cornea 202 due to the transparency of the cornea 202.

The eye 200 further includes a lens 214 (e.g., a crystalline lens) located behind the iris 210 and the pupil 212. The lens 214 is a transparent, flexible, biconvex structure that focuses light onto retina 216, which is a thin layer of tissue lining the back of the eye. The lens 214 is attached to ciliary body 218 by suspensory ciliary ligaments 220 (e.g., Zonules of Zinn), which include fine transparent fibers. The ciliary body 218 adjusts the lens shape for near or distant vision. By way of example, the lens 214, by changing its shape, functions to change the focal distance of the eye 200 so that it can focus on objects at various distances, thus allowing a sharp real image of the object of interest to be formed on the retina 216. This adjustment of the lens 214 is known as accommodation and is similar to the focusing of a photographic camera via movement of its lenses.

The lens 214 has three main parts: a lens capsule 222, a lens epithelium 224, and lens fibers 226. The lens capsule 222 (e.g., capsular bag) forms the outermost layer of the lens 214, and the lens fibers 226 form the bulk of the interior of the lens 214. The cells of the lens epithelium 224, located between the lens capsule 222 and the outermost layer of the lens fibers 226, are found predominantly on the anterior side of the lens but extend posteriorly just beyond the equator. The lens capsule 222 is a smooth, transparent basement membrane that completely surrounds the lens 214. The lens capsule 222 is elastic and thus causes the lens 214 to assume a more globular shape when not under the tension of the lens capsule 222. The lens capsule 222 varies between approximately 2-28 micrometers in thickness, being thickest near the equator and thinnest near the posterior pole.

The eye 200 is an organ that reacts to light for several purposes. By way of example, as a conscious sense organ, the eye allows vision. Rod and cone cells in the retina 216 allow conscious light perception and vision, including color differentiation and the perception of depth. In addition, non-image-forming photosensitive ganglion cells in the retina 216 receive light signals that affect adjustment of the size of the pupil 212, regulation and suppression of the hormone melatonin, and entrainment of the body clock.

The retina 216 includes macula 228, which is a small, central area that provides detailed central vision. The optic nerve 206 transmits visual information from the retina 216 to the brain, e.g., via electrical signals. A layer called choroid 230 between the retina 216 and the sclera 204 is a vascular layer that provides circulation to the retina 216.

Thus, the eye 200 is made up of three layers that enclose three transparent structures. The outermost layer includes the cornea 202 and sclera 204. The middle layer includes the choroid 230, the ciliary body 218, and the iris 210. The innermost layer is the retina 216, which gets its circulation from the vessels of the choroid 230 as well as the retinal vessels, which can be seen within an ophthalmoscope. Within these coats are aqueous humor, vitreous humor 232, and the lens 214. The aqueous humor is a clear fluid that is contained in two areas: an anterior chamber between the cornea 202 and the iris 210 and the exposed area of the lens 214; and the posterior chamber, between the iris 210 and the lens 214. The vitreous humor 232 is a clear, gel-like substance that fills the space between the lens 214 and the retina 216 that helps maintain the shape of the eye 200 and allows light to pass through to the retina 216.

### Real-Time Instrument Position Estimation and Guidance for Ophthalmic Surgery

FIG. 3 depicts an example implementation 300 of the real-time instrument position estimation module 136 of FIG. 1 in greater detail. Where appropriate, reference will be made to components previously introduced in FIGS. 1 and 2.

In the implementation 300 shown in FIG. 3, the object recognition algorithm 140 of the real-time instrument position estimation module 136 receives the intra-op data 116. Specifically, the object recognition algorithm 140 receives the first image feed 126 (e.g., from the first camera 122 of the intra-operative imaging system 118) and the second image feed 128 (e.g., from the second camera 124 of the intra-operative imaging system 118). As explained in more detail above with respect to FIG. 1, the first image feed 126 and the second image feed 128 include images depicting at least a portion of the instrument 106 and a portion of the eye of the patient 104.

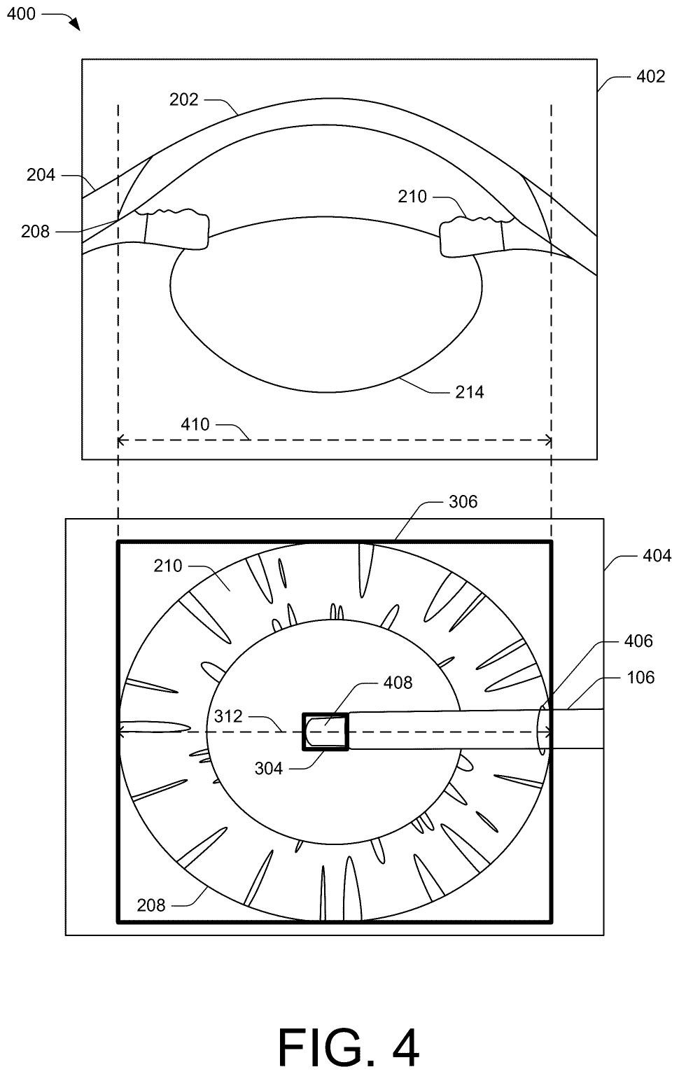

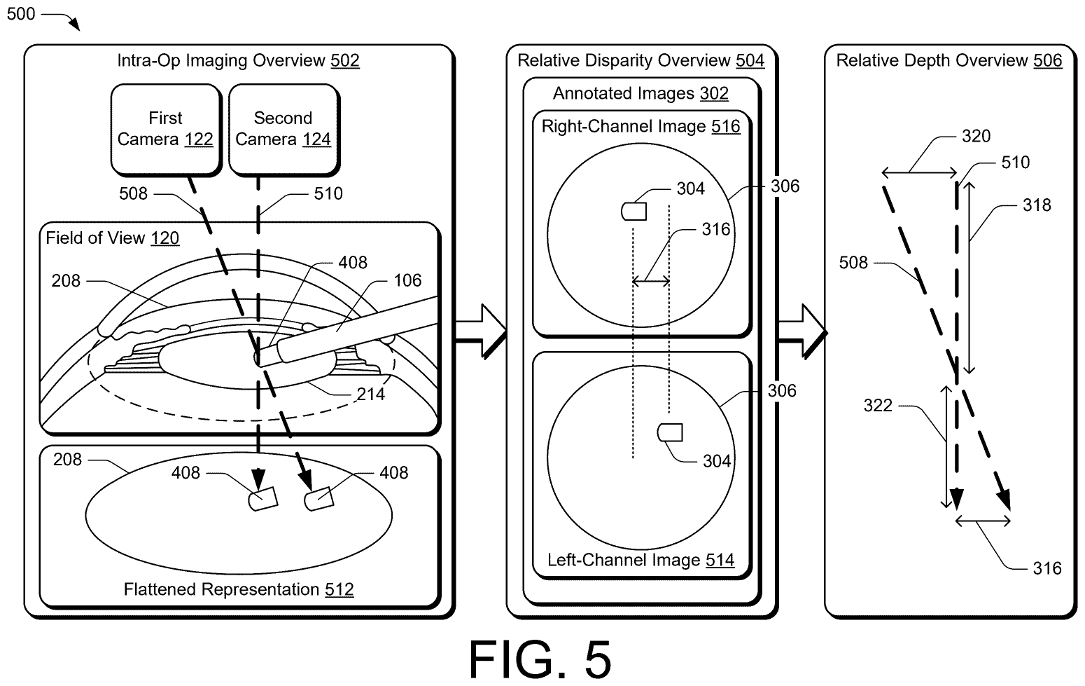

In the depicted example, the object recognition algorithm 140 evaluates the first image feed 126 and the second image feed 128 and generates annotated images 302. The annotated images 302 include a left-channel annotated image (e.g., from the first image feed 126) and a corresponding right-channel annotated image (e.g., from the second image feed 128) and depict an instrument feature 304 and an anatomical feature 306, as will be further described with respect to FIGS. 4 and 5. Although one instrument feature 304 and one anatomical feature 306 are shown in the implementation 300, it is to be appreciated that the annotated images 302 may include more than one instrument feature 304 and/or more than one anatomical feature 306 depending on a type of surgical procedure being performed. In at least one implementation, the annotated images 302 may be displayed as the live image 130.

The instrument feature 304 corresponds to an identified and segmented portion of the instrument 106 that is targeted via the object recognition algorithm 140. Similarly, the anatomical feature 306 corresponds to an identified and segmented portion of the eye 200 that is targeted via the object recognition algorithm 140. As non-limiting examples, the instrument feature 304 may include a tip of the instrument 106, such as a tip of a phacoemulsification instrument used in cataract surgery, a vitrectomy probe used in vitreoretinal surgery, or an implantation device used in glaucoma surgery, just to name a few. The tip, for instance, corresponds to a distal end portion of the instrument 106 that is designed to perform a specific surgical task, such as cutting, emulsifying, aspirating, injecting, and so forth. Non-limiting examples of the anatomical feature 306 include the limbus 208, the pupil 212, the retina 216 or features therein (e.g., blood vessels, the optic disc, etc.), and trabecular meshwork positioned between the cornea 202 and the iris 210, just to name a few.

In at least one implementation, the instrument feature 304 and the anatomical feature 306 are separately indicated in the annotated images 302 with respective bounding boxes and centroids. A bounding box, for example, may correspond to the smallest rectangular box that can completely enclose an object or a geometric shape, while the centroid is the geometric center or average position of all the points in the bounding box. In one or more variations, pixel-level image segmentation is used, where pixels corresponding to the instrument feature 304 are separated from other pixels of the annotated images 302 via an overlay, masking, or another technique that partitions image pixels into segments based on detected edges, for instance.

The position estimation algorithm 142 receives the annotated images 302 and determines the X- and Y-coordinate positions of the instrument feature 304 and/or the anatomical feature 306, represented in FIG. 3 as a spatial location 308. In at least one implementation, the spatial location 308 uses a pixel-based coordinate system that measures a horizontal distance (e.g., number of pixels) from an origin as the X-coordinate and a vertical distance from the origin as the Y-coordinate.

The disparity calculation algorithm 144 receives the spatial location 308 and further receives the pre-op data 114. Although not specifically shown, the disparity calculation algorithm 144 may further receive the annotated images 302. Specifically, the disparity calculation algorithm 144 receives eye measurements 310 of the pre-op data 114, which establish the physical dimensions of the eye 200. By way of example, the eye measurements 310 may be obtained via optical coherence tomography (OCT), ultrasound biometry, or other techniques. Non-limiting examples of the eye measurements 310 include a white-to-white distance of the limbus 208, a curvature of the cornea 202, a thickness of the cornea 202, anterior chamber depth (e.g., a distance from the posterior surface of the cornea 202 to the anterior surface of the lens 214), axial length (e.g., the length of the eye 200 from the anterior surface of the cornea 202 to the retina 216), a thickness of the lens 214, and a thickness of the sclera 204.

In at least on implementation, the disparity calculation algorithm 144 determines a pixel measurement 312, which corresponds to an average measurement of the anatomical feature 306 in image pixels. As an illustrative example where the limbus 208 is the anatomical feature 306, the disparity calculation algorithm 144 may determine, as the pixel measurement 312, the average width of the limbus 208 in pixels based on the width of the limbus 208 (in pixels) in the left-channel annotated image and the width of the limbus 208 (in pixels) in right-channel annotated image. The disparity calculation algorithm 144 may use the pixel measurement 312 and the eye measurements 310 to determine a unit per pixel value 314 of the annotated images 302. By way of example, the unit per pixel value 314 may be calculated by dividing the physical width measurement of the limbus 208 (e.g., in millimeters, as provided in the eye measurements 310) by the pixel measurement 312. The unit per pixel value 314 enables pixel values to be converted to a physical measurement. It is to be appreciated that although examples are provided in millimeters herein, other measurement units may be used for the unit per pixel value 314.

The disparity calculation algorithm 144 may then calculate a relative disparity 316 between the instrument feature 304 and the anatomical feature 306 based on differences in the positions (e.g., the horizontal, X-coordinate positions) of the corresponding objects in the annotated images 302. By way of example, the relative disparity 316 may be calculated as:

$$ΔR = ( ( {X}_{Inst Center}^{Left} - {X}_{Inst Center}^{Right} ) - ( {X}_{Anat Center}^{Left} - {X}_{Anat Center}^{RIght} ) ) * μ$$

where ΔR is the relative disparity 316,

$${X}_{Inst Center}^{Left}$$

is the X-coordinate position of the center (e.g., centroid) of the instrument feature 304 in the left-channel annotated image,

$${X}_{Ins t Center}^{Right}$$

is the X-coordinate position of the center of the instrument feature 304 in the right-channel annotated image,

$${X}_{Anat Center}^{Left}$$

is the X-coordinate position of the center of the anatomical feature 306 in the left-channel annotated image,

$${X}_{Anat Center}^{RIght}$$

is the X-coordinate position of the center of the anatomical feature 306 in the right-channel annotated image, and μ is the unit per pixel value 314. For example, the disparity calculation algorithm 144 may calculate a first disparity as a disparity of the instrument feature 304 between the annotated images 302

$$( e . g . , {X}_{Inst Center}^{Left} - {X}_{Inst Center}^{Right}$$

and a second disparity as a disparity of the anatomical feature 306 between the annotated images 302

$$( e . g . , {X}_{Anat Center}^{Left} - {X}_{Anat Center}^{RIght} ) .$$

The disparity calculation algorithm 144 may then calculate the relative disparity 316 by subtracting the second disparity from the first disparity and converting the resulting value using the unit per pixel value 314.

The depth estimation algorithm 146 receives the relative disparity 316 and further receives the configuration data 134. The configuration data 134 is shown as including a focal length 318 and an optical channel separation 320. The focal length 318 refers to a distance between the optical center of the intra-operative imaging system 118 and the point where parallel rays of light converge at a focal point (or plane) after passing through the intra-operative imaging system 118. The optical channel separation 320 refers to the difference between the viewpoints of the first camera 122 and the second camera 124 (e.g., a stereoscopic base of the intra-operative imaging system 118). The focal length 318 and the optical channel separation 320 may be received in or converted to the same physical measurement unit as the unit per pixel value 314 (e.g., millimeters).

The depth estimation algorithm 146 calculates a relative depth 322 based on the relative disparity 316 and the configuration data 134. The relative depth 322 is the depth of the instrument feature 304 relative to the anatomical feature 306, for instance. By way of example, the relative depth 322 may be calculated as:

$$D = \frac{FL * Δ R}{OCS}$$

where D is the relative depth 322, FL is the focal length 318, OCS is the optical channel separation 320, and ΔR is the relative disparity 316 calculated by the disparity calculation algorithm 144.

As mentioned above, the relative depth 322 provides an indication of where the instrument feature 304 is positioned relative to the anatomical feature 306 in terms of anterior to posterior depth. In at least one implementation, the relative depth 322 is a positive value when the instrument feature 304 is anterior to the anatomical feature 306 and a negative value when the instrument feature 304 is posterior to the anatomical feature 306. In such scenarios, the relative depth 322 may be equal to zero when the instrument feature 304 is coplanar with the anatomical feature 306. In this example, coplanar means in a plane extending in the X and Y directions at a particular depth, but other configurations are possible unless otherwise stated.

Together, the spatial location 308 and the relative depth 322 provide a relative three-dimensional position 324 of the instrument feature 304 with respect to the anatomical feature 306.

The position prediction algorithm 148 receives the relative three-dimensional position 324 and outputs a position prediction 326 based thereon. Although not explicitly shown in FIG. 3, the position prediction 326 may be further based on the spatial location 308. In at least one implementation, the position prediction algorithm 148 uses data from previous image frames of the annotated images 302 to track any change(s) in the relative three-dimensional position 324 of the instrument feature 304 over time. The position prediction 326, for instance, corresponds to an anticipated movement of the instrument feature 304 based on the current relative three-dimensional position 324 compared to preceding relative three-dimensional positions 324. For example, the position prediction 326 may enable the real-time instrument position estimation module 136 to generate preemptive alerts as a part of the surgical guidance 110, such as will be elaborated below.

A guidance generator 328 of the real-time instrument position estimation module 136 generates the surgical guidance 110 based on the position prediction 326 and/or the relative three-dimensional position 324. In general, the position indication 138 and/or the surgical guidance 110 indicates whether the relative three-dimensional position 324 of the instrument feature 304 (e.g., the current relative depth 322 and/or the current spatial location 308) is desired or undesired based on the type of procedure being performed. In at least one implementation, the position indication 138 of the surgical guidance 110 provides the relative three-dimensional position 324 or a portion thereof (e.g., the relative depth 322 and/or the spatial location 308) as numerical value(s) output via the display device 132. Alternatively, or in addition, the guidance generator 328 generates the position indication 138 by comparing the relative depth 322 to one or more thresholds, which may be user adjustable. By way of example, the position indication 138 may include a color-based overlay that indicates the relative depth 322 in comparison to the one or more thresholds. As an illustrative example, the position indication 138 may include a green overlay when the relative depth 322 is within a first depth range (e.g., a targeted depth range of an operation zone for performing the surgical procedure), a yellow overlay when the relative depth 322 is within a second depth range that is immediately outside of the first depth range (e.g., a depth range that is approaching a boundary of the operation zone), and a red overlay when the relative depth 322 is outside of both the first depth range and the second depth range (e.g., outside of the operation zone). As another non-limiting example, additionally or alternatively, the green overlay may indicate that the instrument feature 304 is at least a desired distance from the anatomical feature 306. In this example, the yellow overlay may indicate that the instrument feature 304 is closer to the anatomical feature 306 than the desired distance, but at least a buffer distance from the anatomical feature 306. The red overlay may indicate that the instrument feature 304 is less than the buffer distance from the anatomical feature 306 in this example. In at least one variation, the position prediction 326 is used to output a preemptive alert, such as when crossing outside of the second depth range is anticipated.

It is to be appreciated that the position indication 138 and the surgical guidance 110 may be given in other suitable formats without departing from the spirit or scope of the described techniques. Moreover, a format in which the position indication 138 and/or the surgical guidance 110 are output may be adjusted based on user preferences. Furthermore, in variations, the real-time instrument position estimation module 136 includes more, fewer, or different algorithms than those specifically shown in FIG. 3. For example, in at least one variation, the real-time instrument position estimation module 136 does not include the algorithm 148, and the real-time instrument position estimation module 136 outputs the surgical guidance 110 based on the relative depth 322 and

FIG. 4 depicts a non-limiting example 400 of using pre-op patient measurements to determine physical measurement units from pixel values in the context of cataract surgery.

The non-limiting example 400 includes a pre-op OCT image 402 and in intra-op annotated image 404 (e.g., one of the annotated images 302 of FIG. 3). The pre-op OCT image 402 shows a portion of the eye 200, specifically, the lens 214 positioned posterior to the iris 210 and the limbus 208 where the cornea 202 connects to the sclera 204. Although the non-limiting example 400 is described with respect to cataract surgery, it is to be appreciated that the described techniques are applicable to other types of ophthalmic surgeries or techniques.

The image 404 shows the instrument 106 inserted through a corneal incision 406. In the present example, the instrument 106 is a phacoemulsification instrument, and the instrument feature 304 is a tip of the phacoemulsification instrument, referred to herein as a phaco tip 408. The instrument feature 304 is indicated via a bounding box, although other types of annotations may be used. In the present example, the anatomical feature 306 is the limbus 208. The anatomical feature 306 is also indicated via a corresponding bounding box.

The limbus 208 has a white-to-white distance 410, which is measured via the pre-op OCT image 402. The white-to-white distance 410 is one example of the eye measurements 310 described with respect to FIG. 3, for instance. In the image 404, the limbus 208 has the pixel measurement 312, which corresponds to the white-to-white distance 410. Accordingly, the white-to-white distance 410 and the pixel measurement 312 are used (e.g., by the disparity calculation algorithm 144 of the real-time instrument position estimation module 136) to calculate the unit per pixel value 314, and unit per pixel value 314 is used to calculate the relative disparity 316 and then the relative depth 322 of the phaco tip 408 relative to the limbus 208, as further described herein.

FIG. 5 depicts an illustrative example overview 500 of calculating the relative depth 322 during cataract surgery. Specifically, the illustrative example overview 500 highlights how the disparity of objects represented in left and right imaging channels is used by the real-time instrument position estimation module 136 to estimate the relative depth 322 of the phaco tip 408 with respect to the limbus 208. It is to be appreciated that the illustrative example overview 500 is a simplified, exaggerated view to provide visualization of the values and features discussed herein. Where appropriate, reference is made to components and features previously introduced with respect to FIGS. 1-4.

The illustrative example overview 500 includes an intra-op imaging overview 502, a relative disparity overview 504, and a relative depth overview 506. The intra-op imaging overview 502 depicts the first camera 122 having a first viewing angle 508 of the field of view 120 and the second camera 124 having a second viewing angle 510 of the field of view 120. A flattened representation 512 indicates how the position of the phaco tip 408 within the limbus 208 changes based on the viewing angle. For example, the horizontal (e.g., X-coordinate) position of the phaco tip 408 is different in the first image feed 126 obtained by the first camera 122 relative to the second image feed 128 obtained by the second camera 124.

This is also indicated in the relative disparity overview 504, which depicts the annotated images 302 of the instrument feature 304 (e.g., the phaco tip 408) and the anatomical feature 306 (e.g., the limbus 208). The annotated images 302 include a left-channel image 514 obtained by the first camera 122 and a right-channel image 516 obtained by the second camera 124. The anatomical feature 306 is aligned (e.g., centered) in the left-channel image 514 and the right-channel image 516 in order to offset the disparity of the anatomical feature 306 in the relative disparity calculation performed by the disparity calculation algorithm 144. The distance between the center of the instrument feature 304 in the left-channel image 514 and the right-channel image 516 is the relative disparity 316.

The relative depth overview 506 shows how the configuration data 134 influences the calculation of the relative depth 322 by the depth estimation algorithm 146. By way of example, the relative depth overview 506 shows the first viewing angle 508 and the second viewing angle 510 separated by the optical channel separation 320 and having the focal length 318. The first viewing angle 508 and the second viewing angle 510 converge at a focal plane where the phaco tip 408 is located, for example. As indicated in the intra-op imaging overview 502 and the relative disparity overview 504, the first viewing angle 508 and the second viewing angle 510 result in the relative disparity 316 of the phaco tip 408 between the left-channel image 514 and the right-channel image 516, which is used to calculate the relative depth 322 of the phaco tip 408 with respect to the limbus 208.

Having discussed example details of the techniques for real-time instrument position estimation and guidance for ophthalmic surgery, consider now an example to illustrate usage of the techniques.

### Example Applications

FIG. 6 depicts an example 600 of real-time instrument position estimation and guidance during cataract surgery. Components that function similarly are numbered the same as those previously introduced in FIGS. 1-5.

The example 600 shows a user interface 602 output on the display device 132. The user interface 602 includes the live image 130, which annotated in the present example to depict the instrument feature 304 and the anatomical feature 306. As such, the live image 130 may be one of the annotated images 302 of FIG. 3. The instrument feature 304 and the anatomical feature 306 are annotated via minimum bounding boxes in the example 600. The user interface 602 further depicts the left-channel image 514 and the right-channel image 516, which are shown without annotations in the present example.

In the example 600, the phaco tip 408 is the instrument feature 304, and the anatomical feature 306 is the limbus 208. For instance, the real-time instrument position estimation module 136 identifies, via the object recognition algorithm 140, the phaco tip 408 and the limbus 208. The real-time instrument position estimation module 136 determines, via the position estimation algorithm 142, the spatial location 308 of the instrument feature 304 and the anatomical feature 306 and uses this, via the disparity calculation algorithm 144 to determine the relative disparity 316 between the phaco tip 408 and the limbus 208. For example, the disparity calculation algorithm 144 determines the unit per pixel value 314 based on the white-to-white distance 410 of the limbus 208 in order to output the relative disparity 316 in physical measurement units (e.g., millimeters). The relative disparity 316 is used by the depth estimation algorithm 146, along with the configuration data 134 of the intra-operative imaging system 118, to determine the relative depth 322. The surgical guidance 110 generated based on the relative depth 322, e.g., by the guidance generator 328.

In the example 600, the surgical guidance 110 includes an indication of the relative depth 322, (e.g., “0.33 mm posterior to the limbus”). The relative depth 322 is at least part of the position indication 138, for instance. Moreover, the position indication 138 includes a graph showing the relative depth 322 over time. For instance, during the cataract surgery, the surgeon makes a circular opening (e.g., rhexis) at the front portion of the lens capsule 222 (e.g., the anterior capsule) in a process called capsulorhexis. The phaco tip 408 is inserted through the circular opening and used to emulsify the lens 214, often using a channeling technique. The fragments of the emulsified lens 214 are aspirated.

Although the example 600 shows the surgical guidance 110 as including the relative depth 322, in other implementations, the surgical guidance 110 includes a percentage of the total depth from the plane of the limbus 208 to the annotated images 302 endothelium or to the posterior of the lens capsule 222. For instance, pushing the phaco tip 408 too deep into the lens nucleus risks rupture of the posterior capsular bag. At the same time, if the phaco tip 408 is not deep enough, there is a risk of disrupting the cornea endothelium.

Although not explicitly shown in FIG. 6, the X and Y location (e.g., the spatial location 308) of the phaco tip 408 may also be tracked as a part of the surgical guidance 110. For instance, when the phaco tip 408 is at the same depth plane as the iris 210 and the rhexis, the risk of inadvertently poking the iris or rhexis increases when the phaco tip 408 moves further away from the limbus 208 or pupil 212 centers. As such, in at least one variation, the position indication 138 additionally or alternatively tracks the X and Y locations of the phaco tip 408 with respect to the limbus 208 center and/or the edge of the iris 210. Additionally, or alternatively, the rhexis edge is one of the anatomical features 306 detected and tracked through the procedure, and the distance to rhexis edge is measured. If the shape of the pupil 212 or rhexis can be approximated as a circle, for instance, the surgical guidance 110 may include an alert message or other visual indicator (e.g., a color change or symbol) that is triggered if the phaco tip 408 moves out of a concentric circle defined with a radius that is a certain fraction of the radius of the pupil 212 and/or the rhexis.

As such, in the example 600, the surgical guidance 110, which includes the position indication 138, helps the surgeon judge how to manipulate the phaco tip 408 in order to increase positive patient outcomes.

In this way, the example 600 demonstrates how the real-time instrument position estimation and guidance techniques can be applied to cataract surgery to improve the precision and outcomes of phacoemulsification procedures. By providing real-time depth information and visual guidance, the surgical system 102 enables surgeons to more accurately manipulate the phaco tip 408 within the eye, potentially reducing the risk of complications such as posterior capsule rupture or corneal endothelium damage. The ability to track both the depth and planar position of the phaco tip 408 relative to eye structures allows for more informed decision-making during surgery, potentially leading to better surgical outcomes and reduced variability between procedures.

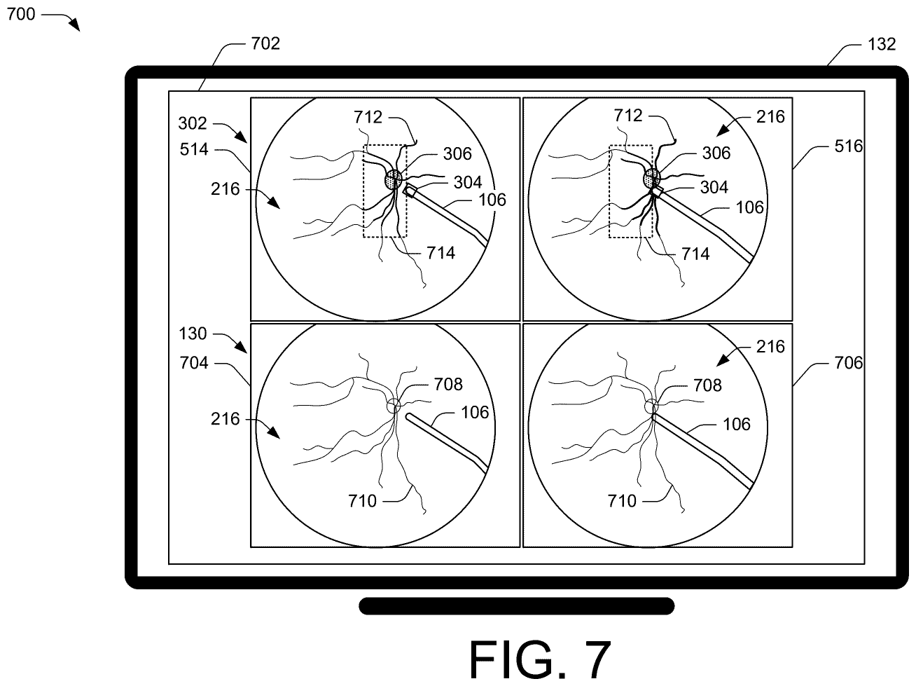

FIG. 7 depicts an example 700 of real-time instrument position estimation and guidance during a vitrectomy as a part of vitreoretinal surgery. Components that function similarly are numbered the same as those previously introduced in FIGS. 1-5.

The example 700 shows a user interface 702 output on the display device 132. The user interface 702 includes two live images 130, including a left-channel live image 704 and a right-channel live image 706, and two corresponding annotated images 302 depicting the retina 216. The left-channel live image 704 and the right-channel live image 706 correspond to the first image feed 126 and the second image feed 128, respectively. In particular, the live images 130 and the annotated images 302 depict optic disc 708 and blood vessels 710 of the retina 216, which are labeled with respect to the live images 130 (and not the annotated images 302) for illustrative clarity.

The annotated images 302 are annotated versions of the live images 130. For example, the user interface 702 depicts the left-channel image 514 and the right-channel image 516, which are annotated in the present example to depict the instrument feature 304 of the instrument 106 and the anatomical feature 306. In the example 700, the instrument 106 is a vitrectomy probe, and the instrument feature 304 is the tip of the vitrectomy probe. The anatomical feature 306 is the optic disc 708. Moreover, the annotated images 302 include additional annotations 712 of the blood vessels 710. As such, the anatomical feature 306 comprise retina features in the present example.

It is to be appreciated that in at least one variation, only the live images 130 or only the annotated images 302 are shown in the user interface 702.

In the example 700, the real-time instrument position estimation module 136 may use, as the eye measurements 310, measurements of the interior surface (e.g., fundus) of the retina 216 (such as its curvature), measurements of the blood vessels 710, a dimension of the optic disc 708, and/or even the distance between the optic disc 708 and the macula 228 (not shown in FIG. 7). As such, the pixel measurement 312 may be converted to physical measurement units based on the physical measurements of the retina 216 or portions thereof. In at least one variation, the unit per pixel value 314 is determined based on dimensions of the instrument 106 in addition to or as an alternative to the eye measurements 310.

In at least one implementation, two target areas in the left-channel image 514 and the right-channel image 516 are defined with a region of interest 714. In at least one implementation, the real-time instrument position estimation module 136 determines the region of interest 714 relative to the instrument feature 304 once the instrument feature 304 is detected. In at least one variation, however, the real-time instrument position estimation module 136 receives the region of interest 714 as user input, such as via intra-op data 116 received through user interaction with the computing system 108. The region of interest 714, for instance, may represent a region of predicted travel of the instrument 106, assuming that the instrument 106 continues to advance while going deeper (e.g., toward the retina 216). Although not explicitly shown in FIG. 7, the surgical guidance 110 may be used by the instrument control module 150 to drive the instrument 106, and specifically the instrument feature 304, toward the region of interest 714.

In at least one implementation, the real-time instrument position estimation module 136 compares the region of interest 714 in the left-channel image 514 and the right-channel image 516 to detect the horizontal shift between the region of interest 714 as the relative disparity 316. The horizontal shift, for instance, may be calculated either through direct image cross-correlation or through aligning the instrument feature 304. This may then be used to calculate the relative depth 322 of the instrument feature 304 to the retina 216.

In this way, the example 700 illustrates how the real-time instrument position estimation and guidance techniques can be applied to vitreoretinal surgery to enhance the precision and outcomes of vitrectomy procedures. By providing real-time 3D position information of the vitrectomy probe tip relative to retinal features such as the optic disc 708 and blood vessels 710, the system enables surgeons to navigate the complex posterior segment of the eye with greater confidence. The ability to define and track regions of interest 714 based on the anatomical feature 306 allows for more targeted and efficient surgical maneuvers, potentially reducing the risk of iatrogenic retinal damage and improving overall surgical outcomes in vitreoretinal procedures.

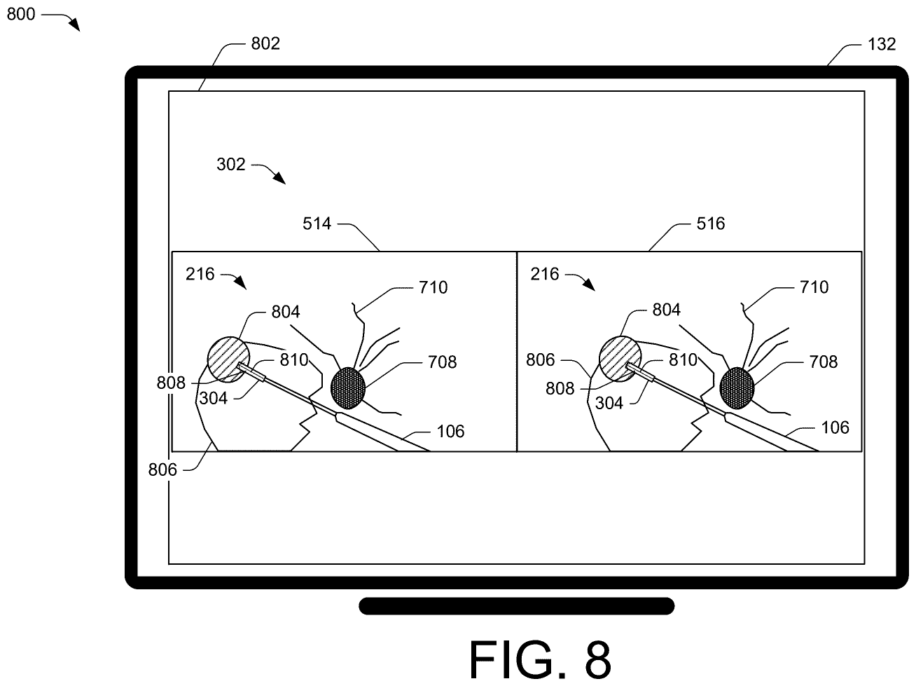

FIG. 8 depicts an example 800 of real-time instrument position estimation and guidance during a subretinal injection as a part of vitreoretinal surgery. Components that function similarly are numbered the same as those previously introduced in FIGS. 1-5.

The example 800 shows a user interface 802 output on the display device 132. The user interface 802 includes the annotated images 302 (e.g., the left-channel image 514 and the right-channel image 516) depicting the retina 216. For example, lighting and imaging conditions during the subretinal injection may obscure visual identification of anatomical features of the retina 216, and so the annotated images 302 may highlight or otherwise distinguish the anatomical features 306 of interest for display on the display device 132.

In the example 800, the annotated images 302 depict the optic disc 708 and blood vessels 710 of the retina 216. The annotated images 302 further depict an injection site 804 and a boundary 806 of a peeled inner limiting membrane of the retina 216 through which the subretinal injection will occur. The optic disc 708, the blood vessels 710, the injection site 804, and the boundary 806 comprise the anatomical features 306 detected by the object recognition algorithm 140, for instance. In this example, the instrument 106 is an injection canula, and the instrument feature 304 is a canula tip 808. Moreover, the example 800 includes an arrow 810 depicting the orientation of the instrument feature 304 (e.g., the canula tip 808).

In at least one implementation, the real-time instrument position estimation module 136 receives, e.g., as intra-op data 116, an indication of the injection site 804. For instance, the indication may be received via user selection of the injection site 804 using an input device to the computing system 108. Moreover, the user (e.g., the surgeon) may further indicate that the injection site 804 is the anatomical feature 306 against which to compute the relative depth 322. In at least one variation, however, the real-time instrument position estimation module 136 automatically determines that the injection site 804 is the anatomical feature 306 against which to compute the relative depth 322 using a pre-programmed workflow for a subretinal injection. For instance, the pre-programmed workflow may prompt the user to define the location of the injection site 804 in the annotated images 302, and, once defined, the real-time instrument position estimation module 136 may automatically calculate the relative depth 322 of the instrument feature 304 (e.g., the canula tip 808) based on the injection site 804 (e.g., the anatomical feature 306) without additional user input.

In at least one implementation, the real-time instrument position estimation module 136 identifies and tracks the instrument feature 304 and the anatomical features 306 in the live annotated images 302. Based on the instrument feature 304, such as the width of the instrument 106, and/or the anatomical features 306, such as the blood vessel spacing, as well as the configuration data 134, the distance between the canula tip 808 and the injection site 804 (e.g., the relative depth 322) may be estimated in physical units, and this estimation may enable robotic control by the instrument control module 150. By way of example, the instrument control module 150 may use coordinates defined in the same physical units to drive the instrument 106.

Thus, although not explicitly shown in FIG. 8, the surgical guidance 110 may be used by the instrument control module 150 to drive the instrument 106, and specifically the instrument feature 304, toward the injection site 804.

In this way, the example 800 showcases how the real-time instrument position estimation and guidance techniques can be applied to subretinal injection procedures to improve accuracy and safety. By providing real-time 3D position information of the injector canula tip 808 relative to the injection site 804 and other retinal features, the surgical system 102 may perform the subretinal injection with enhanced precision. For example, the ability to track the orientation and position of the canula tip, along with the visualization of the boundary 806 of the peeled inner limiting membrane, allows for more controlled and targeted delivery of therapeutic agents to the subretinal space, thus improving the efficacy of subretinal treatments and reducing the risk of complications associated with imprecise injections.

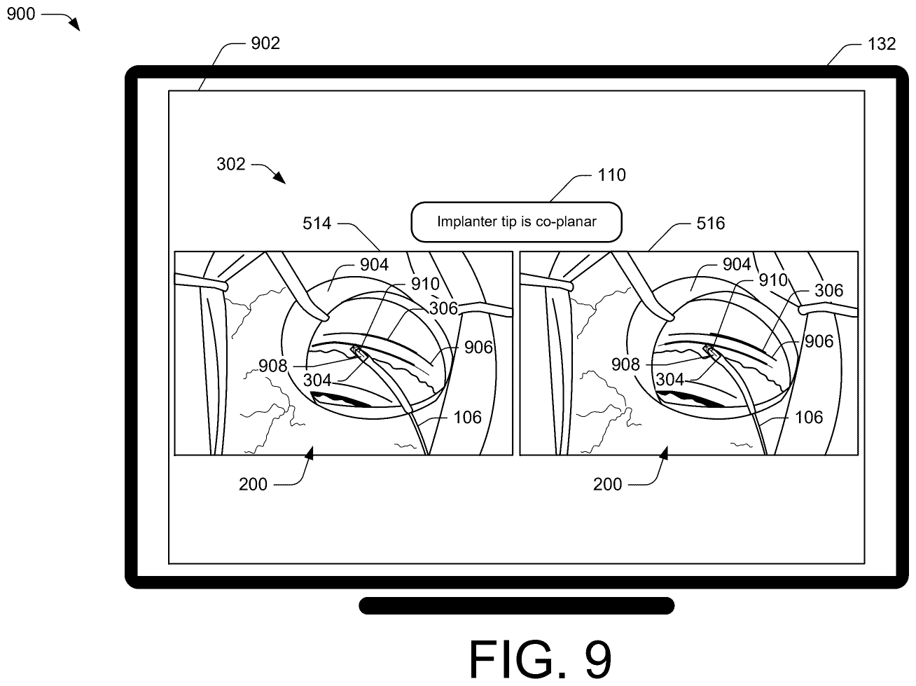

FIG. 9 depicts an example 900 of real-time instrument position estimation and guidance during a minimally invasive glaucoma surgery (MIGS). Components that function similarly are numbered the same as those previously introduced in FIGS. 1-5.

The example 900 shows a user interface 902 output on the display device 132. The user interface 902 includes the annotated images 302 (e.g., the left-channel image 514 and the right-channel image 516) depicting an anterior chamber angle of the eye 200, such as where the cornea 202 and the iris 210 meet. A gonioscopy lens 904 is used to provide a view of anatomical features within the anterior chamber angle, including trabecular meshwork 906. The anatomical feature 306 is the trabecular meshwork 906, which is highlighted in the annotated images 302 via lines overlaid on the real-time images displayed on the display device 132. Moreover, in the example 900, the instrument 106 is a stent implanter configured to implant a stent into the trabecular meshwork 906. As such, the instrument feature 304 is the tip 908 of the stent implanter. Moreover, the example 900 includes an arrow 910 depicting the orientation of the instrument feature 304 (e.g., the tip 908).

Accordingly, in the example 900, the real-time instrument position estimation module 136 detects, as the instrument feature 304, the tip 908 of the stent implanter. The real-time instrument position estimation module 136 further detects the trabecular meshwork 906 as the anatomical feature 306. Based on these detected features, the real-time instrument position estimation module 136 estimates the 3D pointing angle of the tip 908 relative to the trabecular meshwork 906. By way of example, the 3D curvature of the trabecular meshwork 906 at the insertion site can be computed from the detected eye features, particularly the annotation corresponding to the trabecular meshwork 906. Based on the 3D curvature of the trabecular meshwork 906 (e.g., the anatomical feature 306) and the 3D pointing angle of the tip 908 (e.g., the instrument feature 304), the real-time instrument position estimation module 136 generates, as the surgical guidance 110, one or more outputs (e.g., messages) that may be provided to the surgeon regarding 3D alignment information for successful implantation.

For example, the surgical guidance 110 may include information about whether the trabecular meshwork 906 is co-planar to the instrument feature 304. This guidance helps ensure proper alignment for successful stent implantation. The real-time instrument position estimation module 136 may also monitor the process when the stent emerges from the tip 908 of the implanter and is inserted into the trabecular meshwork 906.

In this way, the example 900 demonstrates how the real-time instrument position estimation and guidance techniques can be applied to MIGS procedures to improve the precision and success rate of stent implantation. By providing real-time 3D alignment information, the surgical system 102 enables surgeons to more accurately manipulate the stent implanter, which may increase positive surgical outcomes and reduce a risk of complications.

Having discussed example details of the techniques for real-time instrument position estimation and guidance for ophthalmic surgery, consider now an example procedure to illustrate additional aspects of the techniques.

### Example Procedure

This section describes an example procedure for real-time instrument position estimation and guidance for ophthalmic surgery in one or more implementations. Aspects of the procedure may be implemented in hardware, firmware, or software, or a combination thereof. The procedure is shown as a set of blocks that specify operations performed by one or more devices and are not necessarily limited to the orders shown for performing the operations by the respective blocks. In at least some implementations, at least a portion of the procedure is performed by a suitably configured device, such as the computing system 108 of FIG. 1, by executing instructions stored in a non-transitory computer-readable storage medium.

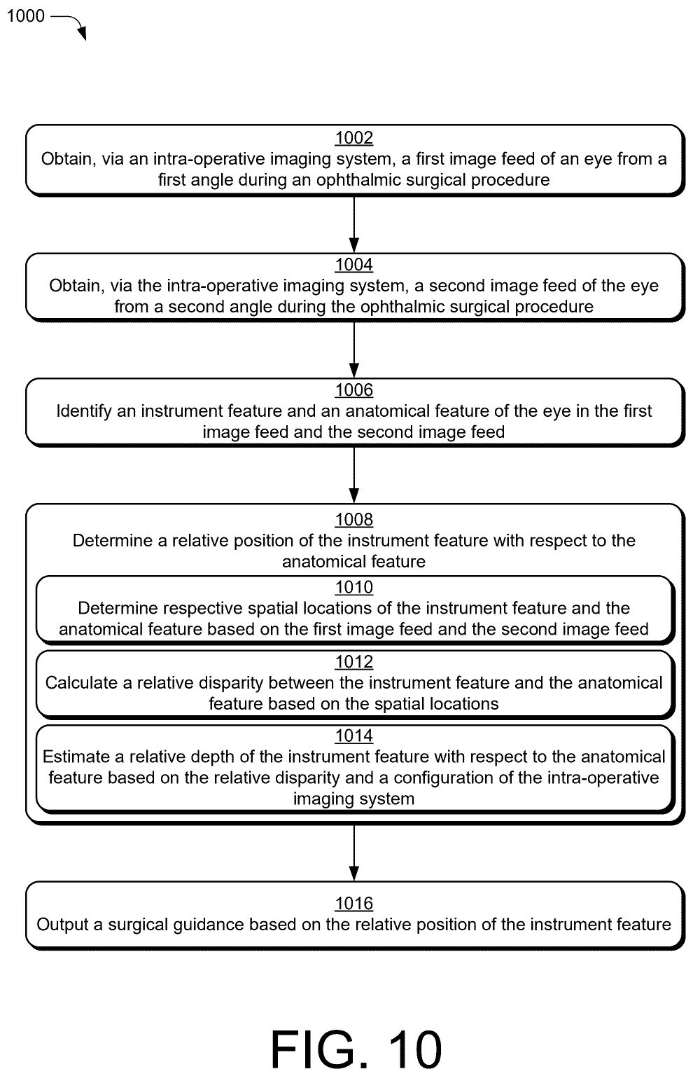

FIG. 10 depicts an example procedure 1000 in which real-time instrument position estimation and guidance for ophthalmic surgery is performed.

A first image feed of an eye is obtained, via an intra-operative imaging system, from a first angle during an ophthalmic surgical procedure (block 1002). By way of example, the first camera 122 of the intra-operative imaging system 118 may obtain the first image feed 126 of the field of view 120 during the ophthalmic surgical procedure.

A second image feed of the eye is obtained, via the intra-operative imaging system, from a second angle during the ophthalmic surgical procedure (block 1004). By way of example, the second camera 124 of the intra-operative imaging system 118 may obtain the second image feed 128 of the field of view 120 during the ophthalmic surgical procedure.

An instrument feature and an anatomical feature of the eye are identified in the first image feed and the second image feed (block 1006). By way of example, the object recognition algorithm 140 of the real-time instrument position estimation module 136 may detect and identify the instrument feature 304 (or features) and the anatomical feature 306 (or features) in the first image feed 126 and the second image feed 128. In at least one implementation, the object recognition algorithm 140 automatically detects the instrument feature 304 and the anatomical feature 306 based on a procedure being performed. In at least one variation, the object recognition algorithm 140 receives user input (e.g., as a part of the input data 112) regarding what is to be detected as the instrument feature 304 and the anatomical feature 306.

A relative position of the instrument feature with respect to the anatomical feature is determined (block 1008). In at least one implementation, the relative position is the relative three-dimensional position 324. The relative three-dimensional position 324 may be determined based at least in part on the intra-op data 116, e.g., the first image feed 126 and the second image feed 128, via the algorithm(s) incorporated in the real-time instrument position estimation module 136, for instance.

In accordance with the techniques described herein, determining the relative position of the instrument feature with respect to the anatomical feature includes determining respective spatial locations of the instrument feature and the anatomical feature based on the first image feed and the second image feed (block 1010). By way of example, the position estimation algorithm 142 may determine the spatial locations 308 of the instrument feature 304 and the anatomical feature 306 using an X- and Y-coordinate system. In at least one implementation, the X- and Y-coordinate system is a pixel-based coordinate system that measures a horizontal distance (e.g., number of pixels) from an origin as the X-coordinate and a vertical distance from the origin as the Y-coordinate.

In accordance with the techniques described herein, determining the relative position of the instrument feature with respect to the anatomical feature further includes calculating a relative disparity between the instrument feature and the anatomical feature based on the spatial location (block 1012). By way of example, the disparity calculation algorithm 144 may calculate the relative disparity 316 between the instrument feature 304 and the anatomical feature 306 based on their respective spatial locations 308. The relative disparity 316, for instance, is the difference between horizontal positions of corresponding points in the first image feed 126 and the second image feed 128.

In at least one implementation, calculating the relative disparity 316 includes calculating, e.g., via the disparity calculation algorithm 144, a unit per pixel value 314 based on pre-operative measurements of the eye (e.g., the eye measurements 310) and a pixel measurement 312 of the anatomical feature 306. For example, the unit per pixel value 314 may be calculated by dividing a physical width measurement of the anatomical feature 306 (e.g., in millimeters, as provided in the eye measurements 310) by the pixel measurement 312. The relative disparity 316 may then be calculated based on a first difference between the respective coordinate positions (e.g., the spatial locations 308) of the instrument feature 304 in the first image feed 126 and the second image feed 128, a second difference between the respective coordinate positions of the anatomical feature 306 in the first image feed 126 and the second image feed 128, and the unit per pixel value 314. Additionally, or alternatively, the disparity calculation algorithm 144 may use a physical measurement of the instrument 106 (e.g., the instrument feature 304) for determining the unit per pixel value 314.

In accordance with the techniques described herein, determining the relative position of the instrument feature with respect to the anatomical feature further includes estimating a relative depth of the instrument feature with respect to the anatomical feature based on the relative disparity and a configuration of the intra-operative imaging system (block 1014). By way of example, the depth estimation algorithm 146 may estimate the relative depth 322 of the instrument feature 304 with respect to the anatomical feature 306 based on the relative disparity 316 and the configuration data 134 of the intra-operative imaging system 118. The configuration data 134 may include a focal length 318 of the first camera 122 and the second camera 124 and an optical channel separation 320 between the first camera 122 and the second camera 124, which provides a geometric arrangement of the first camera 122 and the second camera 124 with respect to the obtained images.

In at least one implementation, the relative depth 322 provides an indication of where the instrument feature 304 is positioned relative to the anatomical feature 306 in terms of anterior to posterior depth. In at least one implementation, the relative depth 322 is a positive value when the instrument feature 304 is anterior to the anatomical feature 306 or a negative value when the instrument feature 304 is posterior to the anatomical feature 306. In such scenarios, the relative depth 322 may be equal to zero when the instrument feature 304 is coplanar with the anatomical feature 306.

Together, the spatial location 308 and the relative depth 322 may provide a relative three-dimensional position 324 of the instrument feature 304 with respect to the anatomical feature 306. This relative three-dimensional position 324 enables precise tracking and guidance of the instrument 106 during various ophthalmic surgical procedures.

A surgical guidance is output based on the relative position of the instrument feature (block 1016). By way of example, the real-time instrument position estimation module 136 may output the surgical guidance 110 based on the relative three-dimensional position 324 of the instrument feature 304 with respect to the anatomical feature 306. The surgical guidance 110 may include a position indication 138 that provides information regarding the position of the instrument 106 (e.g., the 3D position, the depth, or the horizontal and vertical position) relative to the targeted anatomical feature.

In at least one implementation, the position indication 138 includes a visual indicator generated based on the relative position of the instrument feature 304 with respect to at least one threshold that defines a targeted position of the instrument feature 304 relative to the anatomical feature 306. For example, the position indication 138 may include a color-based overlay or another visual overlay that indicates the relative depth 322 in comparison to the at least one threshold. As an illustrative example, the position indication 138 may include a green overlay when the relative depth 322 is within a first depth range (e.g., a targeted depth range of an operation zone for performing the surgical procedure), a yellow overlay when the relative depth 322 is within a second depth range that is immediately outside of the first depth range (e.g., a depth range that is approaching a boundary of the operation zone), and a red overlay when the relative depth 322 is outside of both the first depth range and the second depth range (e.g., outside of the operation zone).

Additionally, or alternatively, the surgical guidance 110 may include numerical values representing the relative three-dimensional position 324 or a portion thereof (e.g., the relative depth 322 and/or the spatial location 308) output via the display device 132. In some implementations, the surgical guidance 110 may further include an instrument adjustment 156 provided to the instrument 106 that comprises a command to adjust one or more of an angle, a depth, a plane, a speed, a directionality, and a parameter setting of the instrument 106.

It is to be appreciated that the procedure 1000 may be adapted for a plurality of different ophthalmic surgical procedures. For example, in a cataract surgery scenario, the instrument feature 304 may be a tip of a phacoemulsification probe (e.g., the phaco tip 408), and the anatomical feature 306 may be the limbus 208. In a vitrectomy scenario, the instrument feature 304 may be a tip of a vitrectomy probe, and the anatomical feature 306 may be a retinal feature, such as the optic disc 708 or blood vessels 710. For a subretinal injection procedure, the instrument feature 304 may the canula tip 808, and the anatomical feature 306 may be a retinal feature, such as an injection site 804. In a minimally invasive glaucoma surgery scenario, the instrument feature 304 may be the tip 908 of a stent implanter, and the anatomical feature 306 may be the trabecular meshwork 906.

By providing real-time instrument position estimation and guidance across various ophthalmic surgical procedures, the procedure 1000 enables more precise and efficient surgeries, which may result in improved patient outcomes and reduced surgeon-to-surgeon variation.

Having described example procedures in accordance with one or more implementations, consider now an example system and device that can be utilized to implement the various techniques described herein.

### Example System and Device

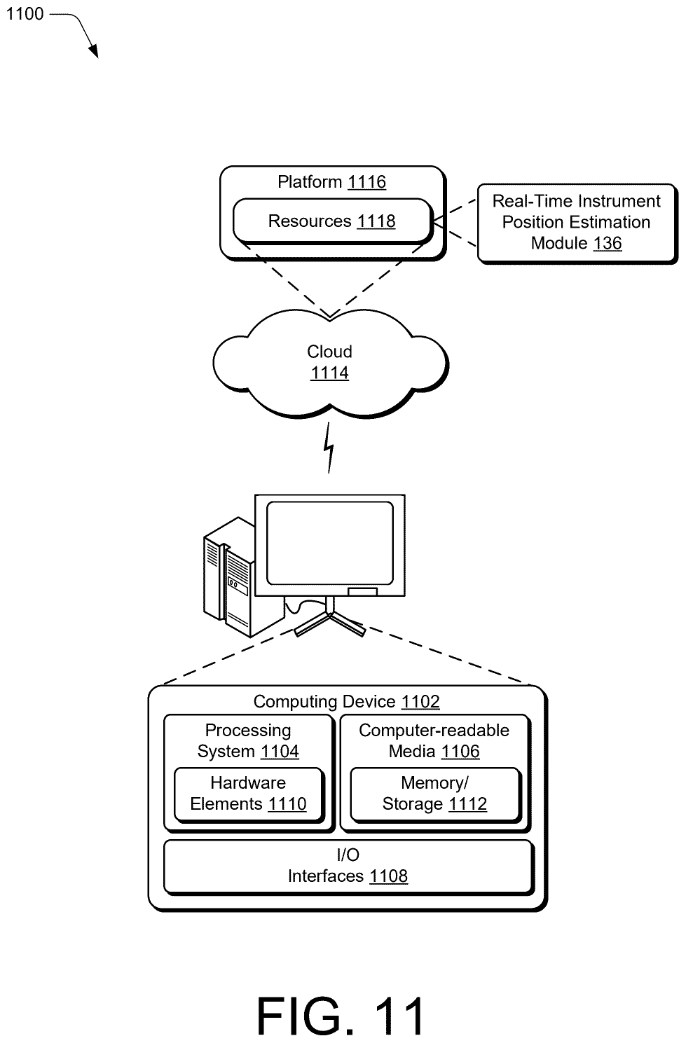

FIG. 11 illustrates an example system generally at 1100 that includes an example computing device 1102 that is representative of one or more computing systems and/or devices that may implement the various techniques described herein. The computing device 1102 may be, for example, a server of a service provider, a device associated with a client (e.g., a client device), an on-chip system, and/or any other suitable computing device or computing system. The computing device 1102 is one example of the computing system 108 of FIG. 1, for example.

The example computing device 1102 as illustrated includes a processing system 1104, one or more computer-readable media 1106, and one or more I/O interfaces 1108 that are communicatively coupled, one to another. Although not shown, the computing device 1102 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 1104 is representative of functionality to perform one or more operations using hardware. Accordingly, the processing system 1104 is illustrated as including hardware elements 1110 that may be configured as processors, functional blocks, and so forth. This may include implementation in hardware as an application specific integrated circuit or other logic device formed using one or more semiconductors. The hardware elements 1110 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable storage media 1106 is illustrated as including memory/storage 1112. The memory/storage 1112 represents memory/storage capacity associated with one or more computer-readable media. The memory/storage 1112 may include volatile media (such as random-access memory (RAM)) and/or nonvolatile media (such as read only memory (ROM), Flash memory, optical disks, magnetic disks, and so forth). The memory/storage 1112 may include fixed media (e.g., RAM, ROM, a fixed hard drive, and so on) as well as removable media (e.g., flash memory, a removable hard drive, an optical disc, and so forth). The computer-readable media 1106 may be configured in a variety of other ways as further described below.

Input/output interface(s) 1108 are representative of functionality to allow a user to enter commands and information to computing device 1102, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 1102 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hardwired device that performs operations based on hardwired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 1102. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media, and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 1102, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 1110 and computer-readable media 1106 are representative of modules, programmable device logic and/or fixed device logic implemented in a hardware form that may be employed in some examples to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 1110. The computing device 1102 may be configured to implement particular instructions and/or functions corresponding to the software and/or hardware modules. Accordingly, implementation of a module that is executable by the computing device 1102 as software may be achieved at least partially in hardware, e.g., through use of computer-readable storage media and/or hardware elements 1110 of the processing system 1104. The instructions and/or functions may be executable/operable by one or more articles of manufacture (for example, one or more computing devices 1102 and/or processing systems 1104) to implement techniques, modules, and examples described herein.

The techniques described herein may be supported by various configurations of the computing device 1102 and are not limited to the specific examples of the techniques described herein. This functionality may also be implemented all or in part through use of a distributed system, such as over a “cloud” 1114 via a platform 1116 as described below.

The cloud 1114 includes and/or is representative of a platform 1116 for resources 1118, which are depicted including the real-time instrument position estimation module 136 in the present example. It is to be appreciated, however, that in other implementations, the real-time instrument position estimation module 136 may run locally on a client server or computer rather than on the platform 1116. The platform 1116 abstracts underlying functionality of hardware (e.g., servers) and software resources of the cloud 1114. The resources 1118 may include applications and/or data that can be utilized while computer processing is executed on servers that are remote from the computing device 1102. Resources 1118 can also include services provided over the Internet and/or through a subscriber network, such as a cellular or Wi-Fi network.

The platform 1116 may abstract resources and functions to connect the computing device 1102 with other computing devices. The platform 1116 may also serve to abstract scaling of resources to provide a corresponding level of scale to encountered demand for the resources 1118 that are implemented via the platform 1116. Accordingly, in an interconnected device example, implementation of functionality described herein may be distributed throughout the system 1100. For example, the functionality may be implemented in part on the computing device 1102 as well as via the platform 1116 that abstracts the functionality of the cloud 1114.

### CONCLUSION

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

What is claimed is:

1. An ophthalmic surgical system comprising:
a first camera to obtain a first image feed of an ophthalmic surgical procedure;
a second camera to obtain a second image feed of the ophthalmic surgical procedure; and
a real-time instrument position estimation module implemented in a non-transitory computer-readable storage medium and configured to perform operations comprising:
determining, in real-time, a relative position of an instrument feature with respect to an anatomical feature of an eye based on the first image feed and the second image feed during the ophthalmic surgical procedure; and
outputting, in real-time, a surgical guidance based on the relative position of the instrument feature with respect to the anatomical feature of the eye during the ophthalmic surgical procedure.

2. The ophthalmic surgical system of claim 1, wherein determining, in real-time, the relative position of the instrument feature with respect to the anatomical feature of the eye based on the first image feed and the second image feed during the ophthalmic surgical procedure comprises:
determining respective spatial locations of the instrument feature and the anatomical feature in the first image feed and the second image feed;
calculating a relative disparity between the instrument feature and the anatomical feature based on the respective spatial locations; and
estimating a relative depth of the instrument feature with respect to the anatomical feature based on the relative disparity and a configuration of the first camera and the second camera.

3. The ophthalmic surgical system of claim 2, wherein determining the respective spatial locations of the instrument feature and the anatomical feature in the first image feed and the second image feed comprises:
identifying the instrument feature and the anatomical feature in the first image feed and the second image feed using a machine learning model that is trained to detect the instrument feature and the anatomical feature in the respective images.

4. The ophthalmic surgical system of claim 2, wherein calculating the relative disparity between the instrument feature and the anatomical feature comprises:
determining respective pixel coordinate positions of the instrument feature and the anatomical feature based on the respective spatial locations;
calculating a unit per pixel value based on pre-operative measurements of the eye and a pixel measurement of the anatomical feature; and
calculating the relative disparity based on a first difference between the respective coordinate positions of the instrument feature in the first image feed and the second image feed, a second difference between the respective coordinate positions of the anatomical feature in the first image feed and the second image feed; and the unit per pixel value.

5. The ophthalmic surgical system of claim 2, wherein the configuration of the first camera and the second camera comprises a focal length and an optical channel separation.

6. The ophthalmic surgical system of claim 1, wherein the relative position comprises one or more of a relative depth of the instrument feature with respect to the anatomical feature of the eye, a relative three-dimensional position of the instrument feature with respect to the anatomical feature of the eye, and a planar distance between the instrument feature and the anatomical feature of the eye.

7. The ophthalmic surgical system of claim 1, wherein the surgical guidance comprises a position indication.

8. The ophthalmic surgical system of claim 7, wherein outputting the surgical guidance further comprises:
generating a visual indicator of the position indication based on the relative position of the instrument feature with respect to at least one threshold that defines a targeted position of the instrument feature relative to the anatomical feature; and
outputting the visual indicator via a display.

9. The ophthalmic surgical system of claim 1, wherein the surgical guidance comprises an instrument adjustment provided to an instrument having the instrument feature, the instrument adjustment comprising a command to adjust one or more of an angle, a depth, a plane, a speed, a directionality, and a parameter setting of the instrument.

10. The ophthalmic surgical system of claim 1, wherein:
the ophthalmic surgical procedure is cataract surgery;
the instrument feature is a tip of a phacoemulsification probe; and
the anatomical feature is the limbus.

11. The ophthalmic surgical system of claim 1, wherein:
the ophthalmic surgical procedure is a vitrectomy;
the instrument feature is a tip of a vitrectomy probe; and
the anatomical feature is a retinal feature.

12. The ophthalmic surgical system of claim 1, wherein:
the ophthalmic surgical procedure is a subretinal injection;
the instrument feature is a tip of an injection canula; and
the anatomical feature is a retinal feature.

13. The ophthalmic surgical system of claim 1, wherein:
the ophthalmic surgical procedure is minimally invasive glaucoma surgery;
the instrument feature is a tip of a stent implanter; and
the anatomical feature is Trabecular meshwork.

14. A method for an ophthalmic surgical procedure, comprising:
obtaining, via an intra-operative imaging system, images of an eye during the ophthalmic surgical procedure, the intra-operative imaging system comprising a first camera and a second camera having different viewpoints of the ophthalmic surgical procedure;
processing the images of the ophthalmic surgical procedure in real-time to determine a relative position of a targeted instrument feature with respect to a targeted anatomical feature of the eye; and
outputting a surgical guidance in real-time based on the relative position of the targeted instrument feature with respect to the targeted anatomical feature of the eye.

15. The method of claim 14, wherein processing the images of the ophthalmic surgical procedure in real-time to determine the relative position of the targeted instrument feature with respect to the targeted anatomical feature of the eye comprises:
detecting, via an object recognition algorithm, the targeted instrument feature and the targeted anatomical feature of the eye in the images of the ophthalmic surgical procedure as the images of the ophthalmic surgical procedure are obtained;
estimating, via a position estimation algorithm, respective spatial locations of the targeted instrument feature and the targeted anatomical feature of the eye in the images of the ophthalmic surgical procedure in response to the detecting;
calculating, via a disparity calculation algorithm, a relative disparity between the targeted instrument feature and the targeted anatomical feature of the eye based on the respective spatial locations and pre-operative eye measurement data; and
estimating, via a depth estimation algorithm, a relative depth of the targeted instrument feature with respect to the targeted anatomical feature of the eye based on the relative disparity, an optical camera separation between the first camera and the second camera, and a focal length of the first camera and the second camera.

16. The method of claim 15, wherein the pre-operative eye measurement data comprise a measurement of the targeted anatomical feature of the eye in a physical measurement unit, and wherein calculating, via the disparity calculation algorithm, the relative disparity between the targeted instrument feature and the targeted anatomical feature of the eye based on the respective spatial locations comprises:
calculating a first disparity of the targeted instrument feature based on the respective spatial locations of the targeted instrument feature in a first image obtained by the first camera and a second spatial location of the targeted instrument feature in a second image obtained by the second camera, the first image and the second image obtained simultaneously;
calculating a second disparity of the targeted instrument feature based on the respective spatial locations of the targeted anatomical feature in the first image and the second image;
calculating the relative disparity in pixel values based on a difference between the first disparity and the second disparity; and
converting the pixel values to the physical measurement unit based on a pixel measurement of the targeted anatomical feature in the images and the measurement of the targeted anatomical feature in the physical measurement unit.

17. The method of claim 15, wherein the object recognition algorithm includes a machine learning model trained to detect the targeted instrument feature and the targeted anatomical feature in the images based on the ophthalmic surgical procedure being performed.

18. The method of claim 14, wherein the surgical guidance comprises an overlay on the images regarding the relative position of the targeted instrument feature with respect to the targeted anatomical feature of the eye and with further respect to an adjustable threshold range.

19. An ophthalmic surgical system comprising:
an intra-operative imaging system to obtain images of an ophthalmic surgical procedure performed on an eye, the intra-operative imaging system comprising a first camera and a second camera; and
a real-time instrument position estimation module implemented in a non-transitory computer-readable storage medium and configured to perform operations comprising:
determining, in real-time as the images are obtained, a relative position of an instrument feature with respect to an anatomical feature of the eye based on the images, pre-operative measurements of the eye, and an arrangement of the first camera and the second camera in the intra-operative imaging system; and
outputting, in real-time as the images are obtained, a surgical guidance based on the relative position of the instrument feature with respect to the anatomical feature of the eye.

20. The system of claim 19, wherein the surgical guidance comprises a visualization of the relative position of the instrument feature with respect to the anatomical feature of the eye.
